(19) United States
(12) Patent Application Publication
(10) Pub. No.: US 2008/0214542 A1
(43) Pub. Date: Sep. 4, 2008

(54) Arylpiperazine Derivatives and their Use as Selective Dopamine D3 Receptor Ligands

(75) Inventors: Marc Capet, Melesse (FR); Denis Danvy, Yvetot (FR); Catherine Dartois, Saint Gregoire (FR); Nicolas Levoin, Mordelles (FR); Marcel Morvan, Pace (FR); Isabelle Berrebi-Bertrand, Pace (FR); Thierry Calmels, Melesse (FR); Philippe Robert, Pace (FR); Jean-Charles Schwartz, Paris (FR); Jeanne-Marie Lecomte, Paris (FR)

Correspondence Address: STITES & HARBISON PLLC, 1199 NORTH FAIRFAX STREET, SUITE 900, ALEXANDRIA, VA, 22314, US

(73) Assignee: BIOPROJET, Paris (FR)

(21) Appl. No.: 11/720,526

(22) PCT Filed: Nov. 29, 2005
(86) PCT No.: PCT/FR05/02964

§ 371 Date: Apr. 11, 2008

(30) Foreign Application Priority Data
Dec. 1, 2004 (FR) 0412763

(51) Int. Cl. A61K 31/496; C07D 403/06; C07D 413/14; A61K 31/454; A61P 25/00; A61K 31/5377; C07D 401/06

(52) U.S. Cl. 514/235.2; 544/373; 544/121; 546/201; 514/254.09; 514/323

(57) ABSTRACT

The invention concerns compounds of general formula (I), a method for preparing same, as well as their use as therapeutic agent.

The present invention relates to new arylpiperazine derivatives, a process for preparation thereof and use thereof as therapeutic agents.

More precisely, the compounds according to the present invention have the property of being selective dopamine D3 receptor ligands.

Numerous arylpiperazine derivatives are already known as dopamine antagonists at the level of its type D2 or D4 receptors, some of which possess neuroleptic properties, or as serotonin antagonists or noradrenaline antagonists.

The patent WO 01/49 679 describes arylpiperazine derivatives exhibiting dopamine antagonist properties on its D3 and D4 receptors; nevertheless these compounds have on the one hand a phenyl at position 4 of the piperazine, this phenyl group being of necessity substituted by a halogen, and on the other hand they have at position 1 of the piperazine an alkylene chain optionally having a carbonyl then a bicycle formed by a nitrogen heterocycle with five or six links fused with a benzene, such as indoline or isoquinoline. Nevertheless, only compounds exhibiting an alkylene chain having 2 to 4 carbon atoms are described.

Moreover, no compound of the isoindoline type is described.

It has now been discovered, totally unexpectedly, that the compounds according to the invention which constitute a new series of arylpiperazine derivatives exhibiting a strong affinity for the dopamine D3 receptor. In contrast to the compounds described in WO 01/49769, the derivatives according to the invention have an indoline cycle, an alkylene chain comprising 5 or 6 carbon atoms and a phenyl group not substituted by a halogen atom. They are moreover selective D3 ligands.

These compounds are useful as medicaments in neuropsychiatry, particularly in the treatment of the psychotic or depressive states, in the treatment of motor disorders such as dyskinesias or essential tremors. Moreover, they are useful in the treatment of dependency upon nicotine, cocaine, alcohol and morphine derivatives and also in order to facilitate breaking of the habit in drug-dependent subjects.

The present invention therefore relates to compounds of formula (I):

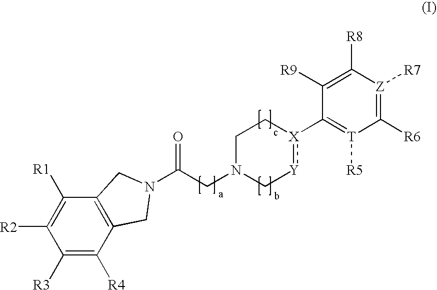

in which:
a=5 or 6;
b and c are identical or different and represent 1 or 2;
X [symbol] Y represents >N—CH₂—, >C═CH—, >CH—CH₂—;
Z and T are identical or different and independently represent a nitrogen or carbon atom;
[symbol] is absent or represents a simple bond, it being understood that when Z and/or T represents a nitrogen atom, [symbol] is absent;
R1, R2, R3 and R4 each independently represent a hydrogen atom or a halogen atom or a hydroxy, alkyl, monofluoroalkyl, polyfluoroalkyl, alkoxy, polyfluoroalkoxy, alkylsulphanyl, polyfluoroalkylsulphanyl, cyano, —OS(O)ₘ-alkyl, —NRR′, —COOR, —COR, —CONRR′, —C(OH)RR′, -alkylC(OH)RR′, -alkylCOOR, -alkylCOR, -alkylCONRR′, -alkylNRR′, -OalkylNRR′, -Oalkyl(NRR′)COOR group or saturated or unsaturated heterocycle or two of the adjacent R1, R2, R3 and R4 are joined to one another to form a hydrocarbon ring or a saturated or unsaturated heterocycle, fused to the phenyl core to which they are attached;
R5, R6, R7, R8 and R9 each independently represent a hydrogen atom or a hydroxy, alkyl, monofluoroalkyl, polyfluoroalkyl, alkoxy, polyfluoroalkoxy, alkylsulphanyl, polyfluoroalkylsulphanyl, cyano, —NRR′, —COOR, —COR, —CONRR′ group or two of the adjacent R5, R6, R7, R8 and R9 are linked to one another in order to form a hydrocarbon ring or a saturated or unsaturated heterocycle, fused to the phenyl core to which they are attached;
   where R, R′, which are identical or different, independently represent a hydrogen atom, an alkyl group optionally substituted by one or several groups chosen from amongst OH, NRR′, OR, COOR or R and R′ are linked to one another in order to form a polymethylene or an oxapolymethylene;
   m=0, 1 or 2;
   it being understood that when T=Z=N, R5 and R7 are absent, R6 and R8 are chosen independently from amongst a hydrogen atom or an alkyl, monofluoroalkyl, polyfluoroalkyl, alkoxy, polyfluoroalkoxy, alkylsulphanyl, polyfluoroalkylsulphanyl, cyano, —COOR, —COR, —CONRR′ group and R9 is chosen from amongst a hydrogen atom or a hydroxy, alkyl, monofluoroalkyl, polyfluoroalkyl, alkoxy, polyfluoroalkoxy, alkylsulphanyl, polyfluoroalkylsulphanyl, —NRR′, —COOR, —COR, —CONRR′ group;
   or R8 and R9 are linked to one another in order to form a hydrocarbon ring or a saturated or unsaturated heterocycle, fused to the phenyl core to which they are attached;
   as well as their stereoisomers or mixtures, their tautomeric forms, their hydrates, solvates, their pharmaceutically acceptable salts and esters.

Preferably a=5.

Preferably b and c represent 1.

Preferably X [symbol] Y represent >N—CH₂—.

Preferably Z=T=C or Z=T=N.

Preferably R1, R2, R3, R4 independently represent a hydrogen or halogen atom or an alkoxy, —OS(O)ₘ-alkyl, cyano, —COOR, —COR, —CONRR′, —C(OH)RR′, -OalkylNRR′ group.

Preferably when T=Z=C, R5, R6, R7, R8, R9 independently represent a hydrogen atom or an alkoxy, alkyl, cyano, polyfluoroalkoxy group, such as a perfluoroalkoxy group, such as trifluoromethoxy, or a polyfluoroalkyl group, such as a perfluoroalkyl group, such as trifluoromethyl, or two of the adjacent R5, R6, R7, R8 and R9 are linked to one another in order to form a hydrocarbon ring or a saturated or unsaturated heterocycle, fused to the phenyl core to which they are attached, such as a benzocycloheptene or benzofuran ring.

Preferably, when T=Z=N, R5 and R7 are absent, R6 and R8 are chosen independently from amongst a hydrogen atom or an alkyl, monofluoroalkyl, polyfluoroalkyl, alkoxy, polyfluoroalkoxy, alkylsulphanyl, polyfluoroalkylsulphanyl, cyano, —COOR, —COR, —CONRR′ group and R9 is chosen from amongst a hydrogen atom or a hydroxy, alkyl, monofluoroalkyl, polyfluoroalkyl, alkoxy, polyfluoroalkoxy, alkylsulphanyl, polyfluoroalkylsulphanyl, —NRR′, —COOR, —COR, —CONRR′ group or R8 and R9 are linked to one another in order to form a hydrocarbon ring or a saturated or unsaturated heterocycle, fused to the phenyl core to which they are attached.

More preferably, the compounds of formula (I) are chosen from amongst the compounds of formula (I′):

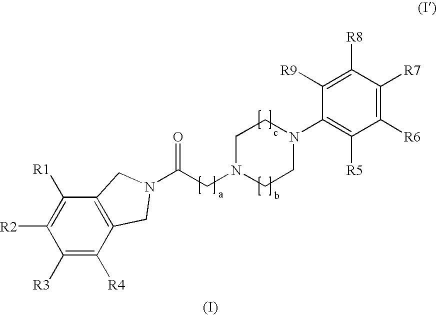

in which a, b and c, R1, R2, R3 and R4, R5, R6, R7, R8 and R9 are as defined in formula (I),
as well as their stereoisomers or mixtures, their tautomeric forms, their hydrates, solvates, their pharmaceutically acceptable salts and esters.

The compounds of formula (I) may be in particular chosen from amongst:
1-(5-methoxyisoindolin-2-yl)-6-[4-(3-trifluoromethylphenyl)piperazin-1-yl]hexan-1-one
1-(5-methanesulphonyloxyisoindolin-2-yl)-6-[4-(3-trifluoromethylphenyl)-piperazin-1-yl]hexan-1-one
1-(5-cyanoisoindolin-2-yl)-6-[4-(3-trifluoromethylphenyl)-piperazin-1-yl]-hexan-1-one hydrochloride
1-isoindolin-2-yl-6-[4-(3-trifluoromethoxyphenyl)piperazin-1-yl]hexan-1-one
1-isoindolin-2-yl-6-[4-(6,7,8,9-tetrahydro-5H-benzocylohepten-2-yl)piperazin-1-yl]hexan-1-one
1-(4-fluoroisoindolin-2-yl)-6-[4-(3-trifluoromethylphenyl)piperazin-1-yl]hexan-1-one
1-(5-fluoroisoindolin-2-yl)-6-[4-(3-trifluoromethylphenyl)piperazin-1-yl]hexan-1-one
1-(5-isopropoxyisoindolin-2-yl)-6-[4-(3-trifluoromethylphenyl)piperazin-1-yl]hexan-1-one
1-(4-chloroisoindolin-2-yl)-6-[4-(3-trifluoromethylphenyl)piperazin-1-yl]hexan-1-one
1-(6,7-dihydro-5H-[1,3]dioxolo[4,5-f]isoindol-6-yl)-6-[4-(3-trifluoromethylphenyl)piperazin-1-yl]hexan-1-one
1-(5-hydroxymethylisoindolin-2-yl)-6-[4-(3-trifluoromethylphenyl)-piperazin-1-yl]hexan-1-one
1-(5-methoxycarbonylisoindolin-2-yl)-6-[4-(3-trifluoromethylphenyl)piperazin-1-yl]hexan-1-one hydrochloride
1-(5-carboxyisoindolin-2-yl)-6-[4-(3-trifluoromethylphenyl)piperazin-1-yl]hexan-1-one
1-[5-(2-hydroxyethylaminocarbonyl)isoindolin-2-yl]-6-[4-(3-trifluoromethylphenyl)piperazin-1-yl]hexan-1-one
1-(5-morpholinocarbonylisoindolin-2-yl)-6-[4-(3-trifluoromethylphenyl)-piperazin-1-yl]hexan-1-one
1-(5-(pyrrolidin-1-ylcarbonylisoindolin-2-yl)-6-[4-(3-trifluoromethylphenyl)-piperazin-1-yl]hexan-1-one
1-(5-piperidin-1-ylcarbonylisoindolin-2-yl)-6-[4-(3-trifluoromethylphenyl)-piperazin-1-yl]hexan-1-one
1-[5-(2-dimethylamino-ethylaminocarbonyl)isoindolin-2-yl]-6-[4-(3-trifluoromethylphenyl)piperazin-1-yl]hexan-1-one
1-[5-(4,5-dihydrooxazol-2yl)isoindolin-2-yl]-6-[4-(3-trifluoromethylphenyl)-piperazin-1-yl]hexan-1-one
1-(5-morpholinoylmethylisoindolin-2-yl)-6-[4-(3-trifluoromethylphenyl)piperazin-1-yl]hexan-1-one hydrochloride
1-[5-(pyrrolidin-1-ylmethyl)isoindolin-2-yl]-6-[4-(3-trifluoromethylphenyl)-piperazin-1-yl]hexan-1-one
1-[5-(dimethylaminomethyl)isoindolin-2-yl]-6-[4-(3-trifluoromethylphenyl)-piperazin-1-yl]hexan-1-one
1-[5-(2-piperidinoethoxy)isoindolin-2-yl]-6-[4-(3-trifluoromethylphenyl)-piperazin-1-yl]hexan-1-one
1-[5-(3-piperidinopropoxy)isoindolin-2-yl]-6-[4-(3-trifluoromethylphenyl)-piperazin-1-yl]hexan-1-one
1-[5-(3-pyrrolidinopropoxy)isoindolin-2-yl]-6-[4-(3-trifluoromethylphenyl)-piperazin-1-yl]hexan-1-one
1-(5-acetylisoindolin-2-yl)-6-[4-(3-trifluoromethylphenyl)piperazin-1-yl]hexan-1-one
1-[5-(1-hydroxy-1-methylethyl)isoindolin-2-yl]-6-[4-(3-trifluoromethylphenyl)piperazin-1-yl]hexan-1-one
1-[5-(1-hydroxyethyl)isoindolin-2-yl]-6-[4-(3-trifluoro-methylphenyl)piperazin-1-yl]hexan-1-one hydrochloride
1-(5-aminocarbonylisoindolin-2-yl)-6-[4-(3-trifluoromethylphenyl)-piperazin-1-yl]hexan-1-one
1-isoindolin-2-yl-6-[4-(3-trifluoromethylphenyl)-piperazin-1-yl]hexan-1-one hydrochloride
1-isoindolin-2-yl-6-[4-(2-methoxyphenyl)piperazin-1-yl]hexan-1-one
1-isoindolin-2-yl-6-[4-(6,7,8,9-tetrahydro-5H-benzocylohepten-1-yl)piperazin-1-yl]hexan-1-one
1-isoindolin-2-yl-6-[4-(2,3-dimethylphenyl)piperazin-1-yl]hexan-1-one
1-isoindolin-2-yl-6-[4-benzofuran-7-ylpiperazin-1-yl]hexan-1-one
1-isoindolin-2-yl-6-[4-(2-cyano-3-trifluoromethylphenyl)piperazin-1-yl]hexan-1-one
1-isoindolin-2-yl-6-(4-indan-5ylpiperazin-1-yl)hexan-1-one
1-isoindolin-2-yl-6-(4-indan-4ylpiperazin-1-yl)hexan-1-one
1-isoindolin-2-yl-6-[4-(5,6,7,8-tetrahydronaphthalen-1-yl)piperazin-1-yl]hexan-1-one
1-isoindolin-2-yl-6-[4-(3-methanesulphonyloxyphenyl)piperazin-1-yl]hexan-1-one
1-isoindolin-2-yl-6-[4-(3,4-dihydro-2H-benzo[b][1,4]dioxepin-6-yl)piperazin-1-yl]hexan-1-one
1-isoindolin-2-yl-6-[4-(3-trifluoromethyl-5-cyanophenyl)piperazin-1-yl]hexan-1-one
1-isoindolin-2-yl-6-[4-(5,6,7,8-tetrahydronaphthalen-2-yl)-piperazin-1-yl]hexan-1-one
1-isoindolin-2-yl-6-[4-(3-isopropoxyphenyl)piperazin-1-yl]hexan-1-one
1-isoindolin-2-yl-6-[4-(2-cyano-3-methylphenyl)piperazin-1-yl]hexan-1-one
1-isoindolin-2-yl-6-[4-(2-cyano-3-isopropoxy)piperazin-1-yl]hexan-1-one
1-(5,6-dimethoxyisoindolin-2-yl)-6-[4-(3-trifluoromethylphenyl)piperazin-1-yl]hexan-1-one
1-isoindolin-2-yl-6-[4-(2-cyanophenyl)piperazin-1-yl]hexan-1-one
1-isoindolin-2-yl-6-[4-(2-methyl-phenyl)piperazin-1-yl]hexan-1-one
1-isoindolin-2-yl-6-(4-phenyl-3,6-dihydro-2H-pyridin-1-yl)hexan-1-one
1-isoindolin-2-yl-6-[4-(3-trifluoromethylphenyl)-3,6-dihydro-2H-pyridin-1-yl]hexan-1-one
1-isoindolin-2-yl-6-[4-(3-trifluoromethylphenyl)piperidin-1-yl]hexan-1-one
1-isoindolin-2-yl-6-[4-(2-cyano-3-methylphenyl)-3,6-dihydro-2H-pyridin-1-yl]hexan-1-one
1-isoindolin-2-yl-6-[4-(2-cyano-3-methylphenyl)piperidin-1-yl]hexan-1-one,
   as well as their stereoisomers or their mixtures, their tautomeric forms, their hydrates, solvates, their pharmaceutically acceptable salts, free forms and esters.

More preferably, the compounds of formula (I) may be chosen from amongst:
1-(5-methoxyisoindolin-2-yl)-6-[4-(3-trifluoromethylphenyl)piperazin-1-yl]hexan-1-one
1-(5-cyanoisoindolin-2-yl)-6-[4-(3-trifluoromethylphenyl)-piperazin-1-yl]-hexan-1-one hydrochloride
1-(5-fluoroisoindolin-2-yl)-6-[4-(3-trifluoromethyl-phenyl)piperazin-1-yl]hexan-1-one
1-(6,7-dihydro-5H-[1,3]dioxolo[4,5-f]isoindol-6-yl)-6-[4-(3-trifluoromethylphenyl)piperazin-1-yl]hexan-1-one
1-(5-methoxycarbonylisoindolin-2-yl)-6-[4-(3-trifluoromethylphenyl)piperazin-1-yl]hexan-1-one hydrochloride
1-[5-(3-piperidinopropoxy)isoindolin-2-yl]-6-[4-(3-trifluoromethylphenyl)piperazin-1-yl]hexan-1-one
1-isoindolin-2-yl-6-[4-(5,6,7,8-tetrahydro-naphthalen-1-yl)piperazin-1-yl]hexan-1-one
1-isoindolin-2-yl-6-[4-(3-trifluoromethylphenyl)-3,6-dihydro-2H-pyridin-1-yl]hexan-1-one
1-isoindolin-2-yl-6-[4-(3-trifluoromethyl-phenyl)piperidin-1-yl]hexan-1-one
   as well as their stereoisomers or their mixtures, their tautomeric forms, their hydrates, solvates, their pharmaceutically acceptable salts, free forms and esters.

According to the present invention, the alkyl radicals represent saturated hydrocarbon radicals with a straight or branched chain and having 1 to 20 carbon atoms, preferably 1 to 5 carbon atoms.

When these radicals are linear, reference may be made in particular to methyl, ethyl, propyl, butyl, pentyl, hexyl, octyl, nonyl, decyl, dodecyl, hexadecyl and octadecyl radicals.

When the radicals are branched or substituted by one or several alkyl radicals, reference may be made in particular to isopropyl, tert-butyl, 2-ethylhexyl, 2-methylbutyl, 2-methylpentyl, 1-methylpentyl and 3-methylheptyl radicals.

The alkoxy radicals according to the present invention are radicals of formula —O-alkyl, the alkyl being as defined above.

Amongst the halogen atoms, reference is made more particularly to fluorine, chlorine, bromine and iodine atoms, preferably fluorine.

The alkenyl radicals represent hydrocarbon radicals with a straight or linear chain, and comprise one or several ethylenic unsaturations. Amongst the alkenyl radicals, reference may be made in particular to allyl or vinyl radicals.

The alkynyl radicals represent hydrocarbon radicals, with a straight or linear chain, and comprise one or several acetylenic unsaturations. Amongst the alkynyl radicals, reference may be made in particular to acetylene.

The cycloalkyl radical is a mono-, bi- or tricyclic saturated or partially unsaturated non-aromatic hydrocarbon radical with 3 to 10 carbon atoms, such as in particular cyclopropyl, cyclopentyl, cyclohexyl or adamantyl, as well as the corresponding rings containing one or several unsaturations.

Aryl designates a mono- or bicyclic aromatic hydrocarbon system with 6 to 10 carbon atoms.

Amongst the aryl radicals, reference may be made in particular to the phenyl or naphthyl radical, more particularly substituted by at least one halogen atom.

Amongst the -alkylaryl radicals, mention may be made in particular of the benzyl or phenetyl radical.

The heteroaryl radicals designate aromatic systems comprising one or several heteroatoms chosen from amongst nitrogen, oxygen or sulphur, mono- or bicyclic, with 5 to 10 carbon atoms. Amongst the heteroaryl radicals, mention may be made of pyrazinyl, thienyl, oxazolyl, furazanyl, pyrrolyl, 1,2,4-thiadiazolyl, naphthyridinyl, pyridazinyl, quinoxalinyl, phthalazinyl, imidazo[1,2-a]pyridine, 1′imidazo[2,1-b]thiazolyl, cinnolinyl, triazinyl, benzofurazanyl, azaindolyl, benzimidazolyl, benzothienyl, thienopyridyl, thienopyrimidinyl, pyrrolopyridyl, imidazopyridyl, benzoazaindole, 1,2,4-triazinyl, benzothiazolyl, furanyl, imidazolyl, indolyl, triazolyl, tetrazolyl, indolizinyl, isoxazolyl, isoquinolinyl, isothiazolyl, oxadiazolyl, pyrazinyl, pyridazinyl, pyrazolyl, pyridyl, pyrimidinyl, purinyl, quinazolinyl, quinolinyl, isoquinolyl, 1,3,4-thiadiazolyl, thiazolyl, triazinyl, isothiazolyl, carbazolyl, as well as the corresponding groups resulting from their fusion or from the fusion with the phenyl core.

The expression “pharmaceutically acceptable salts” refers to relatively non-toxic inorganic and organic acid addition salts, and basic addition salts, of the compounds of the present invention. These salts may be prepared in situ during the final isolation and purification of the compounds. In particular, the acid addition salts may be prepared by separately reacting the purified compound in its purified form with an organic or inorganic acid and isolating the salt thus formed. Examples of acid addition salts include the salts bromohydrate, chlorohydrate, sulphate, bisulphate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulphamates, malonates, salicylates, propionates, methylenebis-beta-hydroxynaphthoates, gentisic acid, isethionates, di-p-toluoyltartrates, methanesulphonates, ethanesulphonates, benzenesulphonates, p-toluenesulphonates, cyclohexyl sulphamates and quinateslaurylsulphonate, and analogues. (See for example S. M. Berge et al. “Pharmaceutical Salts” J. Pharm. Sci, 66: p. 1-19 (1977) which is incorporated herein by reference). The acid addition salts can also be prepared by separately reacting the purified compound in its acid form with an organic or inorganic base and isolating the salt thus formed. The acid addition salts include amine and metal salts. Suitable metal salts include sodium, potassium, calcium, barium, zinc, magnesium and aluminium salts. Sodium and potassium salts are preferred. The suitable basic inorganic addition salts are prepared from metal bases which comprise sodium hydride, sodium hydroxide, potassium hydroxide, calcium hydroxide, aluminium hydroxide, lithium hydroxide, magnesium hydroxide, zinc hydroxide. Suitable basic amine addition salts are prepared from amines which have a sufficient alkalinity to form a stable salt, and preferably comprise the amines which are often used in medical chemistry because of their low toxicity and their acceptability for medical use: ammonia, ethylenediamine, N-methyl-glucamine, lysine, arginine, ornithine, choline, N,N′-dibenzylethylenediamine, chloroprocaine, diethanolamine, procaine, N-benzyl-phenethylamine, diethylamine, piperazine, tris(hydroxymethyl)-aminomethane, tetramethylammonium hydroxide, triethylamine, dibenzylamine, ephenamine, dehydroabiethylamine, N-ethyl-piperidine, benzylamine, tetramethylammonium, tetraethylammonium, methylamine, dimethylamine, trimethylamine, ethylamine, basic amino acids, for example lysine and arginine, dicyclohexylamine, and analogues.

The invention also relates to stereoisomers or mixtures thereof, tautomic forms, hydrates, solvates, pharmaceutically acceptable salts and esters of the compounds of formula (I).

Since the compounds of the invention of formula (I) as defined above possess a sufficiently acidic function or a sufficiently basic function or both, they can include the corresponding pharmaceutically acceptable salts of organic or mineral acid or of organic or mineral base.

The compounds of general formula (I) may be prepared by application or adaptation of any method which is known per se and/or within the scope of the person skilled in the art, in particular those described by Larock in Comprehensive Organic Transformations, VCH Pub., 1989, or by application or adaptation of the process described in the following examples.

According to another object, the present invention therefore also relates to the process of preparation of the compounds of formula (I) described above comprising the step of coupling of the corresponding compounds of formula (II) and (III) according to the following diagram:

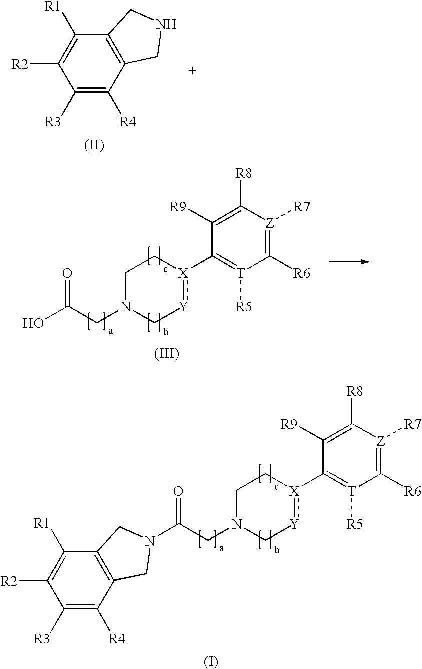

where, in the formula (II) and (III), R1, R2, R3, R4, R5, R6, R7, R8, R9, X, Y, Z, T, . . . , [symbol] , a, b and c have the same meaning as in formula (I).

Generally this condensation is effected with the aid of peptidic coupling agents such as a carbodiimide (dicyclohexylcarbodiimide, diisopropylcarbodiimide, 3-ethyl-1-(3-dimethylaminopropyl)carbodiimide for example) in the presence of a catalyst (1-hydroxybenzotriazole, 4-dimethyl-aminopyridine) in an inert solvent such as ethyl acetate, acetonitrile, dichloromethane or a mixture of these solvents at a temperature between −10° C. and the boiling temperature of the reaction medium. This condensation can also be carried out by first activating the acid in the form of acid chloride with the aid of a chlorination agent (oxalyl chloride, thionyl chloride, phosgene, diphosgene, triphosgene, phosphorus oxychloride for example), in the form of symmetrical anhydride or mixed anhydride with a chloroformate (isobutyl, ethyl or isopropyl chloroformate for example), in the form of phenol ester (paranitrophenol, parachlorophenol or pentafluorophenol for example) or in the form of hydroxyheterocyclic ester (N-hydroxysuccinimide or 1-hydroxybenzotriazole for example). The acid thus activated is then opposed to the compound of formula (II) optionally in the presence of an organic or mineral base (triethylamine, 4-dimethylaminopyridine, potassium carbonate, sodium carbonate, potassium hydrogen carbonate, sodium hydrogen carbonate) in an inert solvent such as ethyl acetate, acetonitrile, dichloromethane or a mixture of these solvents at a temperature between −10° C. and the boiling temperature of the reaction medium.

The derivatives of formula (II) in which R1, R2, R3 and R4 have the same meaning as in formula (I) are either commercially available or prepared using the method described in the literature (EP 0343560, Syn. Commun. (1995), 3255, Bioorg. Med. Chem. Lett. (2001), 11, 685-688).

The acids of formula (III) in which R5, R6, R7, R8, R9, a, b and c have the same meaning as in formula (I) may be prepared from the esters (IV) in which R5, R6, R7, R8, R9, X, Y, Z, T, . . . , [symbol] , a, b and c have the same meaning as in formula (I) and R10 represents an alkyl or a benzyl, optionally substituted.

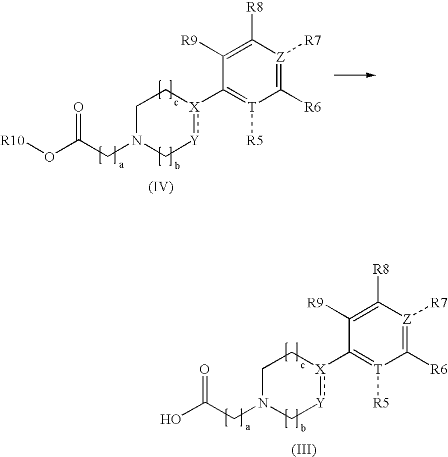

This reaction can be carried out in the presence of an aqueous solution of inorganic base (soda, potash, lithia) optionally in the presence of oxygenated water and an organic cosolvent such as an alcohol (methanol, ethanol for example) or an ether (dioxan, tetrahydrofuran) or a mixture of these solvents. This reaction is carried out at a temperature between −20° C. and the boiling temperature of the reaction medium. When R10 represents a benzyl, optionally substituted, the acid can be obtained by hydrogenolysis by means of dihydrogen or by transfer (formic acid optionally in the presence of triethylamine, ammonium formate, cyclohexene, cyclohexadiene for example) in the presence of a transition metal (palladium for example) in an organic solvent such as an alcohol (methanol, ethanol, isopropanol), an ester (ethyl acetate, isopropyl acetate) or a halogenated solvent (chloroform for example) at a temperature between 10° C. and 150° C. and at a pressure between 1 and 100 bars. When R10 represents a benzyl substituted by one or several alkoxys, the acid can also be obtained by oxidation with the aid of cerium nitrate or dichlorodicyanobenzoquinone.

The esters of formula (IV) in which R5, R6, R7, R8, R9, X, Y, Z, T . . . , [symbol] , a, b and c have the same meaning as in formula (I) and R10 represents an alkyl or a benzyl, optionally substituted, may be prepared by condensation of the esters of formula (V) in which a=5 or 6, R10 represents an alkyl or a benzyl, optionally substituted, and Hal represents a halogen atom, preferably bromine with amines of formula (VI) in which R5, R6, R7, R8, R9, X, Y, Z, T, . . . , [symbol] , b and c have the same meaning as in formula (I).

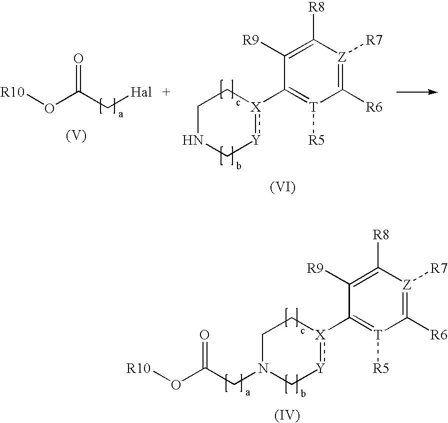

This reaction can be carried out in the presence of an inorganic base (potassium carbonate, caesium carbonate) and in the presence or absence of a catalytic quantity of potassium iodide in a solvent such as acetonitrile, N,N-dimethylformamide, the acetonitrile at a temperature between 15° C. and the boiling temperature of the reaction medium.

The cyclic amines of formula (VI) in which R5, R6, R7, R8, R9, Z, T, . . . , [symbol] , b and c have the same meaning as in formula (I) and X=N, Y=C may be prepared by dialkylation of anilines of formula (VII) in which R5, R6, R7, R8, R9, Z, T, . . . have the same meaning as in formula (I) and of an amine of formula (VIII) in which b and c have the same meaning as in formula (I) and Hal represents a halogen atom, preferably chlorine:

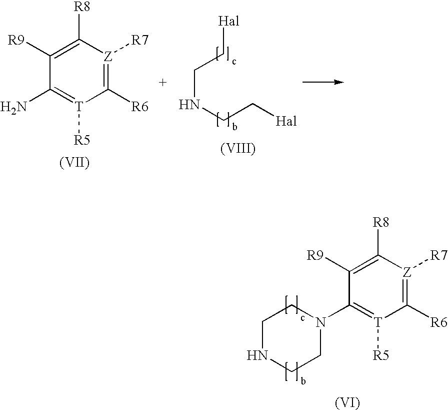

This reaction can be carried out by heating in an aromatic solvent (toluene, chlorobenzene for example) or in an alcohol (ethanol, butanol for example).

The cyclic amines of formula (VI) in which R5, R6, R7, R8, R9, X, Y, Z, T, . . . , [symbol] , b and c have the same meaning as in formula (I) can also be prepared by substitution of haloaromatics, preferably fluoroaromatics of formula (VII) in which Hal represents a halogen atom, preferably fluorine, Z, T, R5, R6, R7, R8 and R9, . . . , have the same meaning as in formula (I) when this latter has an electroattractive group with a diamine of formula (IX) in which b and c have the same meaning as in formula (I)

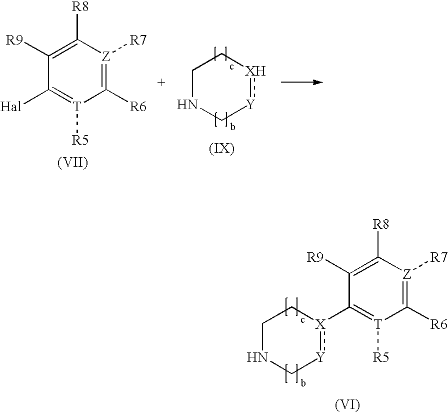

Generally this reaction is carried out by heating in an organic solvent.

The cyclic amines of formula (VI) can also be prepared by haloaromatic substitution, the halogen preferably being an iodine, a bromine or a chlorine or a pseudohaloaromatic, the pseudohalogen being an arylsulphonate or an alkylsulphonate (paratoluenesulphonate, mesylate, triflate for example) of formula (VII).

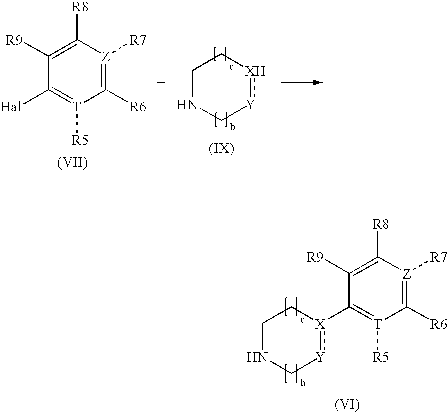

This reaction can optionally be catalysed by transition metals.

The derivatives of formula (I) may also be obtained from cyclic amine derivatives of formula (VI) in which R5, R6, R7, R8, R9, X, Y, Z, T, . . . , [symbol] , b and c have the same meaning as in formula (I) and halogenated derivatives of formula (X) in which R1, R2, R3, R4 and a have the same meaning as in formula (I) and Hal represents a halogen, the chosen halogen preferably being a chlorine or bromine atom:

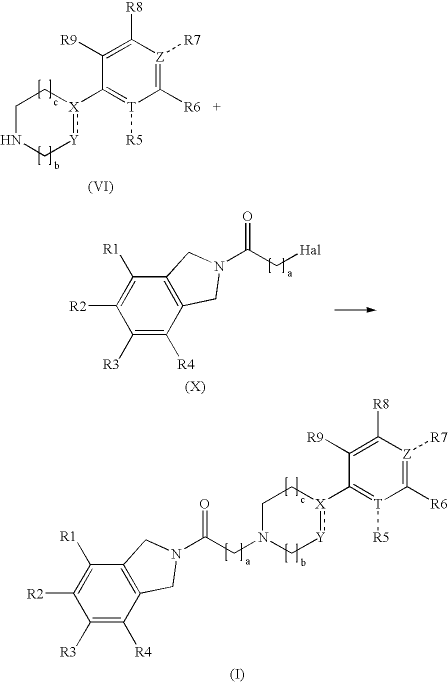

The substitution is carried out in the presence of an inorganic base such as a carbonate or a hydrogen carbonate (potassium carbonate, caesium carbonate for example), in an organic solvent such as acetonitrile, N,N-dimethylormamide or dimethysulphoxide at a temperature between 0° C. and the boiling temperature of the reaction medium.

The derivatives of formula (X) in which R1, R2, R3, R4 and a have the same meaning as in formula (I) and Hal represents a halogen, the chosen halogen preferably being a chlorine or bromine atom, may be prepared from derivatives of isoindoline of formula (XI) in which R1, R2, R3 and R4 have the same meaning as in formula (I) and from an acid of formula (XI) in which the halogen is a chlorine or bromine atom and a=5 or 6:

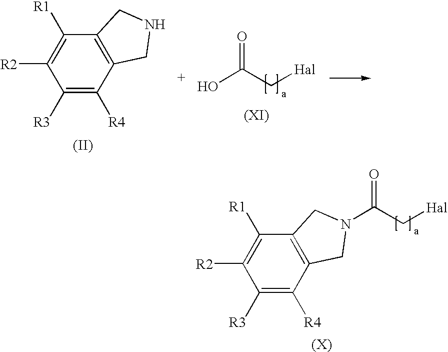

Generally this condensation is carried out with the aid of peptidic coupling agents such as a carbodiimide (dicyclohexylcarbodiimide, diisopropylcarbodiimide, 3-ethyl-1-(3-dimethylaminopropyl)carbodiimide for example) in the presence of a catalyst (1-hydroxybenzotriazole, 4-dimethyl-aminopyridine) in an inert solvent such as ethyl acetate, acetonitrile, dichloromethane or a mixture of these solvents at a temperature between −10° C. and the boiling temperature of the reaction medium. This condensation can also be carried out by first activating the acid in the form of acid chloride with the aid of a chlorination agent (oxalyl chloride, thionyl chloride, phosgene, diphosgene, triphosgene, phosphorus oxychloride for example), in the form of symmetrical anhydride or mixed anhydride with a chloroformate (isobutyl, ethyl or isopropyl chloroformate for example), in the form of phenol ester (paranitrophenol, parachlorophenol or pentafluorophenol for example) or in the form of hydroxyheterocyclic ester (N-hydroxysuccinimide or 1-hydroxybenzotriazole for example). The acid thus activated is then opposed to the compound of formula (II) optionally in the presence of an organic or mineral base (triethylamine, 4-dimethyl-aminopyridine, potassium carbonate, sodium carbonate, potassium hydrogen carbonate, sodium hydrogen carbonate) in an inert solvent such as ethyl acetate, acetonitrile, dichloromethane or a mixture of these solvents at a temperature between −10° C. and the boiling temperature of the reaction medium.

The reaction intermediates of formula V, VII, VIII, IX, XI are commercially available or may be prepared by the person skilled in the art by application or adaptation of methods which are known per se.

The process according to the invention may also comprise the subsequent step of isolation of the products of formula (I) obtained.

In the reactions described here, it may be necessary to protect the reactive functional groups, for example the hydroxy, amino, imino, thio, carboxy groups, when they are desired in the final product, in order to avoid their undesirable participation in the reactions. The traditional protective groups may be used in accordance with standard practice, for examples see T. W. Greene and P. G. M. Wuts in Protective Groups in Organic Chemistry, John Wiley and Sons, 1991; J. F. W. McOmie in Protective Groups in Organic Chemistry, Plenum Press, 1973.

The compound thus prepared may be recovered from the reaction mixture by traditional means. For example, the compounds may be recovered by distilling the solvent from the reaction mixture or if necessary after distillation of the solvent from the solution mixture, by pouring the remainder into water followed by extraction with an organic solvent which is immiscible in water, and by distilling the solvent from the extract. Moreover, if desired the product can be purified again by various techniques, such as recrystallisation, reprecipitation or various chromatographic techniques, in particular column chromatography or preparatory thin layer chromatography.

It will be appreciated that the compounds which are useful according to the present invention can contain asymmetric centres. These asymmetric centres may be independently in R or S configuration. It will be apparent to the person skilled in the art that certain compounds which are useful according to the invention can also present a geometric isomerism. It should be understood that the present invention comprises individual geometric isomers and stereoisomers and mixtures thereof, including racemic mixtures, of compounds of formula (I) above. This type of isomers may be separated from their mixtures by the application or the adaptation of known processes, for example chromatographic techniques or of recrystallisation techniques, or they are prepared separately from the appropriate isomers of their intermediates.

For the purposes of this text it is understood that the tautomeric forms are included in the reference to a given group, for example thio/mercapto or oxo/hydroxy.

The acid addition salts are formed with the compounds which are useful according to the invention in which a basic function such as an amino, alkylamino or dialkylamino group is present. The acid addition salts which are pharmaceutically acceptable, that is to say non-toxic, are preferred. The selected salts are chosen in an optimal manner to be compatible with the usual pharmaceutical vehicles and adapted for oral or parenteral administration. The acid addition salts of the compounds which are useful according to this invention may be prepared by reaction of the free base with the appropriate acid, by the application or the adaptation of known processes. For example, the acid addition salts of the compounds which are useful according to this invention may be prepared either by dissolving the free base in water or in an alcoholic aqueous solution or suitable solvents containing the appropriate acid and isolating the salt by evaporating the solution, or by reacting the free base and the acid in an organic solvent, in which case the salt separates out directly or can be obtained by concentration of the solution. The acids suitable for use in the preparation of these salts include hydrochloric acid, hydrobromic acid, phosphoric acid, sulphuric acid, various organic carboxylic and sulphonic acids, such as acetic acid, citric acid, propionic acid, succinic acid, benzoic acid, tartaric acid, fumaric acid, mandelic acid, ascorbic acid, malic acid, methanesulphonic acid, toluenesulphonic acid, fatty acids, adipate, alginate, ascorbate, aspartate, benzenesulphonate, benzoate, cyclopentene propionate, digluconate, dodecylsulphate, bisulphate, butyrate, lactate, laurate, lauryl sulphate, malate, hydroiodide, 2-hydroxyethanesulphonate, glycerophosphate, picrate, pivalate, pamoate, pectinate, persulphate, 3-phenylpropionate, thiocyanate, 2-naphthalene-sulphonate, undecanoate, nicotinate, hemisulphate, heptonate, hexanoate, camphorate, camphor sulphonate and others.

The acid addition salts of the compounds which are useful according to this invention may be regenerated from the salts by the application or the adaptation of known processes. For example, the parent compounds which are useful according to the invention may be regenerated from their acid addition salts by treatment with an alkali, for example an aqueous sodium bicarbonate solution or an aqueous ammonia solution.

The compounds which are useful according to this invention may be regenerated from their basic addition salts by the application or the adaptation of known processes. For example, the parent compounds which are useful according to the invention may be regenerated from their basic addition salts by treatment with an acid, for example hydrochloric acid.

The basic addition salts may be formed when the compound which is useful according to the invention containing a carboxyl group, or a sufficiently acid bioisosteric. The bases which can be used for preparing the basic addition salts preferably comprise those which, when they are associated with a free acid, produce pharmaceutically acceptable salts, that is to say salts of which the cations are not toxic for the patient in the pharmaceutical doses of the salts, such that the beneficial inhibiting effects inherent in the free base are not negated by the secondary effects attributable to the cations. The pharmaceutically acceptable salts, comprising those which are derivatives of the alkaline earth metal salts, within the scope of the invention comprise those which are derivatives of the following bases: sodium hydride, sodium hydroxide, potassium hydroxide, calcium hydroxide, aluminium hydroxide, lithium hydroxide, magnesium hydroxide, zinc hydroxide, ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxy-methyl)aminomethane, tetramethylammonium hydroxide and analogues.

The compounds which are useful according to the present invention can be easily prepared, or formed during the process of the invention, in the form of solvates (for example hydrates). The hydrates of the compounds which are useful according to the present invention can be easily prepared by the recrystallisation of an aqueous/organic solvent mixture, using organic solvents such as dioxan, tetrahydrofuran or methanol.

The basic products or the reagents used are commercially available and/or may be prepared by the application or the adaptation of known processes, for example processes such as described in the Reference Examples or their obvious chemical equivalents.

According to the present invention, the compounds of formula (I) have a selective D3 receptor ligand activity.

The present invention also relates to pharmaceutical compositions comprising a compound according to the invention with a pharmaceutically acceptable vehicle or excipient.

Preferably, the said composition contains an effective quantity of the compound according to the invention.

According to another object, the present invention also relates to the use of compounds of general formula (I) for the preparation of pharmaceutical compositions intended to act as dopamine D3 receptor ligands. Preferably, the said ligand is a D3 antagonist; even more preferably a select if D3 antagonist.

According to another object, the present invention also relates to the use of compounds of general formula (I) for the preparation of pharmaceutical compositions intended to prevent and/or treat a neuropsychiatric disease or any therapeutic disease causing the intervention of the dopamine D3 receptor. The said diseases are chosen preferably from amongst drug dependency, sexual problems, motor problems, Parkinson's disease, psychosis or a psychotic state, depression or drug dependency.

According to the invention, drug dependency is understood to mean any state linked to the breaking of a habit, abstinence and/or to the detoxification of a subject dependent upon any agent, in particular therapeutically active agents, such as morphine derivatives, and/or drugs such as cocaine, heroin, or even alcohol and/or nicotine.

According to the invention, sexual problems are understood in particular to be impotence, in particular male impotence.

According to the invention, the said prevention and/or treatment of Parkinson's disease is preferably a complementary treatment of Parkinson's disease.

According to the invention, motor problems are understood in particular to be essential or iatrogenic dyskinesias, and/or essential or iatrogenic tremors.

According to another object, the present invention also relates to the above-mentioned therapeutic methods of treatment comprising the administration of a compound according to the invention to a patient who needs it.

Preferably, the said composition is administered to a patient who needs it.

The pharmaceutical compositions according to the invention may be present in forms intended for administration by the parenteral, oral, rectal, permucous or percutaneous route.

They will therefore be present in the form of solutes or injectable suspensions or multi-dose phials, in the form of coated or uncoated tablets, sugar-coated pills, capsules, gelatin capsules, pills, cachets, powders, rectal suppositories or capsules, solutions or suspensions, for percutaneous use in a polar solvent, for permucous use.

The excipients which are suitable for such administrations are the derivatives of cellulose or of microcrystalline cellulose, alkaline earth carbonates, magnesium phosphate, starches, the modified starches, lactose for solid forms.

For rectal use, cocoa butter or polyethyleneglycol stearates are the preferred excipients.

For parenteral use, water, aqueous solutes, physiological serum, isotonic solutes are the most conveniently used vehicles.

The posology may vary within substantial limits (0.5 mg to 1000 mg) as a function of the therapeutic indication and of the administration route, as well as the age and weight of the subject.

The following examples illustrate the invention, but without limiting it. The starting products used are products which are known or prepared according to known modes of operation.

The percentages are expressed by weight, unless stated otherwise.

### EXAMPLES

### Description of the Examples

The melting points are measured on a Büchi B-545 appliance.

The NMR spectra of the proton are recorded on a 250.13 MHz Bruker AC 250 device. The chemical displacements are expressed in ppm. For the multiplicity of the signals, the following abbreviations are used: s=singlet, d=doublet, t=triplet, q=quadruplet, m=multiplet and ms=solid. The coupling constants are expressed in Hertz (Hz).

The thin layer chromatography is carried out on a silica plate.

### Example 1

### Preparation of 1-(5-methoxyisoindolin-2-yl)-6-[4-(3-trifluoromethylphenyl)piperazin-1-yl]hexan-1-one

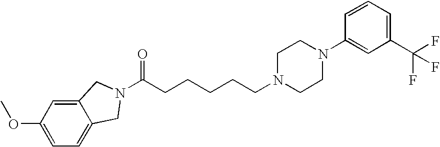

### Step a: Preparation of 6-[4-(3-trifluoromethylphenyl)piperazin-1-yl]ethyl hexanoate

A mixture of 23 g (0.1 mol) of 1-(3-trifluoromethylphenyl)piperazine, 150 mL of acetonitrile, a catalytic quantity of potassium iodide, 13.8 g (0.1 mol) of potassium carbonate and 22.3 g (0.1 mol) of ethyl 6-bromo-hexanoate is brought to reflux for 24 hours.

The reaction medium is concentrated in a vacuum and is taken up with 100 mL of diethyl oxide. The organic phase is washed with 50 mL of water, dried on magnesium sulphate, filtered and concentrated in a vacuum. In this way 37.2 g of a viscous oil is obtained which is used just as it is in the subsequent syntheses.

Rf: 0.55 (ethyl acetate)

NMR ¹H (CDCl₃): 7.35 (t, 1H, J=7.5 Hz); 7.2 to 7.0 (ms, 3H); 4.15 (q, 2H, J=7.5 Hz); 3.3 to 3.15 (ms, 4H); 2.65 to 2.5 (ms, 4H); 2.4 (t, 2H, J=7.5 Hz); 3.2 (t, 2H, J=7.5 Hz); 1.8 to 1.5 (ms, 4H); 1.5 to 1.3 (ms, 2H); 1.25 (t, 3H, J=7.5 Hz)

### Step b: Preparation of 6-[4-(3-trifluoromethyl-phenyl)piperazin-1-yl]hexanoic acid chlorohydrate

A solution of 16.6 g (50 mmol) of 6-ethyl[4-(3-trifluoromethylphenyl)piperazin-1-yl]hexanoate in 200 mL ethanol is stirred and cooled to approximately 5° C. After addition of 50 mL (50 mmol) of a normal aqueous soda solution the reaction medium is stirred during one night at ambient temperature.

Ethanol is concentrated in a vacuum. The residual aqueous phase is washed with 30 mL of diethyl oxide, cooled and acidified with 100 mL of an aqueous solution of 0.5 N of hydrochloric acid. The precipitate formed is filtered, washed with diethyl oxide and dried in a vacuum at 50° C. on phosphorus pentoxide. In this way 9.5 g of a white solid is obtained.

Melting point: 250° C.

NMR ¹H (DMSO d₆): 10.9 (s wide, 1H); 7.65 to 7.4 (ms, 1H); 7.3 to 7.15 (ms, 2H); 7.15 to 7.05 (ms, 1H); 4.1 to 3.7 (ms, 2H); 3.65 to 3.3 (ms, 2H); 3.3 to 3.1 (ms, 2H); 3.1 to 2.8 (ms, 4H); 2.2 (t, 2H, J=7.5 Hz); 1.85 to 1.6 (ms, 2H); 1.6 to 1.4 (ms, 2H); 1.4 to 1.2 (ms, 2H)

### Step c: Preparation of 1-(5-methoxyisoindolin-2-yl)-6-[4-(3-trifluoromethylphenyl)piperazin-1-yl]hexan-1-one

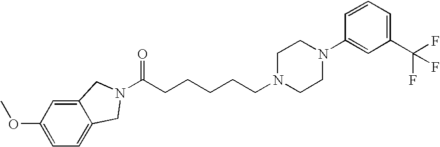

0.344 g (1 mmol) 6-[4-(3-trifluoromethylphenyl)piperazin-1-yl]hexanoic acid, 0.149 g (1.1 mmol) hydroxy-benzotriazole, 0.287 g (1 mmol) 3-ethyl-1-(3-dimethylaminopropyl)carbodiimide hydrochloride and 8 mL of dichloromethane stabilised on amylene are introduced in succession into a flask. This solution is stirred for 10 minutes at ambient temperature then 0.15 g (1 mmol) 5-methoxyisoindoline and 0.31 mL (1.5 mmol) of triethylamine are added.

After stirring at ambient temperature for one night, the reaction medium is diluted with ethyl acetate and washed twice with water. The organic phase is dried on magnesium sulphate, filtered and concentrated in a vacuum. The oily residue obtained is chromatographed on a cartridge containing 5 g of silica, the elution being carried out with a dichloromethane/methanol mixture 98/2 then 95/5. After concentration of the elution fractions, the residue is stirred with diisopropyl oxide. The precipitate is then filtered and dried in a vacuum. In this way 0.16 g of 1-(5-methoxyisoindolin-2-yl)-6-[4-(3-trifluoromethylphenyl)-piperazin-1-yl]hexan-1-one is obtained in the form of a solid.

Melting point: 92° C.

NMR ¹H (CDCl₃): 7.3 (t, 1H, J=7.5 Hz); 7.25 to 7.0 (ms, 4H); 6.9 to 6.8 (ms, 2H); 4.8 (s, 2H); 4.75 (s, 2H); 3.8 (s, 3H); 3.3 to 3.15 (ms, 4H); 2.8 to 2.55 (ms, 4H); 2.55 to 2.2 (ms, 2H); 2.4 (t, 2H, J=7.5 Hz); 1.9 to 1.35 (ms, 6H)

5-methoxyisoindoline can be prepared from 3,4-dimethylanisole using the technique described in EP 0343560.

### Example 2

### Preparation of 1-(5-methanesulphonyloxyisoindolin-2-yl)-6-[4-(3-trifluoromethylphenyl)piperazin-1-yl]hexan-1-one

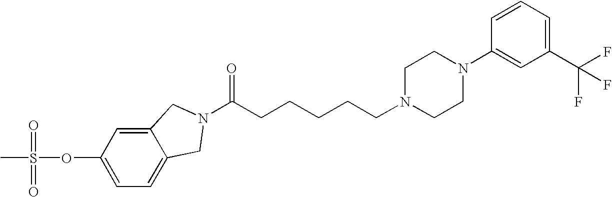

Operating as in Example 1, step c, but starting from 5-methane-sulphonyloxyisoindoline, 1-(5-methanesulphonyloxyisoindolin-2-yl)-6-[4-(3-trifluoromethylphenyl)piperazin-1-yl]-hexan-1-one is obtained in the form of a solid.

Melting point: 109° C.

NMR ¹H (CDCl₃): 7.3 to 7.20 (ms, 3H); 7.2 to 7.0 (ms, 4H); 4.8 (s wide, 4H); 3.4 to 3.2 (ms, 4H); 3.15 (s, 3H); 2.75 to 2.55 (ms, 4H); 2.55 (t wide, 2H, J=7.5 Hz); 2.4 (t, 2H, J=7.5 Hz); 1.9 to 1.5 (ms, 4H); 1.5 to 1.3 (ms, 2H)

5-methanesulphonyloxyisoindoline can be prepared according to the process described in Bioorg. Med. Chem. Lett. (2001), 11, 685-688.

### Example 3

### Preparation of 1-(5-cyanoisoindolin-2-yl)-6-[4-(3-trifluoromethylphenyl)piperazin-1-yl]-hexan-1-one hydrochloride

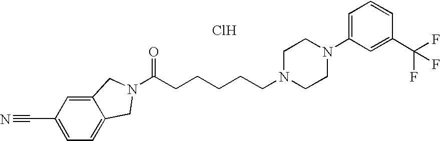

Operating as in Example 1, step c, but starting from 5-cyanoisoindoline the product is obtained in the form of a base. The oily residue is dissolved in diethyl oxide. A saturated solution of hydrogen chloride in diethyl oxide is added. The precipitate is filtered and dried in a vacuum. In this way 1-(5-cyanoisoindolin-2-yl)-6-[4-(3-trifluoromethylphenyl)piperazin-1-yl]-hexan-1-one hydrochloride is obtained in the form of a solid.

Melting point: 155-160° C.

NMR ¹H (DMSO d₆): 10.9 (t wide, 1H); 7.8 (s, 1H); 7.75 (d, 1H, J=7.5 Hz); 7.6 to 7.3 (ms, 2H); 7.25 to 7.15 (ms, 2H); 7.1 (d, 1H, J=7.5 Hz); 5.2 (s, 4H); 4.85 (d, 2H, J=9.5 Hz); 4.65 (d, 2H, J=9.5 Hz); 3.9 (d, 2H, 10 Hz); 3.5 (d, 2H, J=10 Hz); 3.35 to 2.95 (ms, 2H); 2.3 (t, 2H, J=7.5 Hz); 1.9 to 1.7 (ms, 2H); 1.7 to 1.5 (ms, 2H); 1.45 to 2.2 (ms, 2H)

5-cyanoisoindoline can be prepared according to the process described in Bioorg. Med. Chem. Lett. (2001), 11, 685-688.

### Example 4

### Preparation of 1-isoindolin-2-yl-6-[4-(3-trifluoromethoxyphenyl)-piperazin-1-yl]hexan-1-one

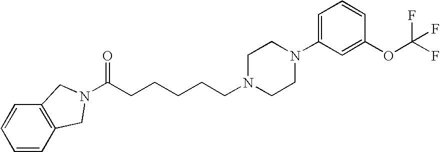

### Step a: Preparation of 6-[4-(3-trifluoromethoxyphenyl)piperazin-1-yl]ethyl hexanoate

Operating as in Example 1, step a, but starting from 2.46 g (3-trifluoromethoxyphenyl)piperazine and 2.23 g ethyl 6-bromohexanoate, 3.5 g ethyl 6-[4-(3-trifluoromethoxyphenyl)piperazin-1-yl]hexanoate is obtained in the form of a solid used as such in the subsequent steps.

### Step b: Preparation of 6-[4-(3-trifluoromethoxyphenyl)piperazin-1-yl]hexanoic acid

Operating as in Example 1, step b, but starting from 3.5 g of ethyl 6-[4-(3-trifluoromethoxyphenyl)piperazin-1-yl]hexanoate, 2.9 g 6-[4-(3-trifluoromethoxyphenyl)piperazin-1-yl]hexanoic acid is obtained in the form of a white solid used as such in the subsequent steps.

### Step c: Preparation of 1-isoindolin-2-yl-6-[4-(3-trifluoromethoxyphenyl)-piperazin-1-yl]hexan-1-one

Operating as in Example 1, step c, but starting from 0.142 g isoindoline and 0.36 g 6-[4-(3-trifluoromethoxyphenyl)piperazin-1-yl]hexanoic acid, 0.125 g 1-isoindolin-2-yl-6-[4-(3-trifluoromethoxyphenyl)piperazin-1-yl]hexan-1-one is obtained in the form of a white solid.

Melting point: 89° C.

NMR ¹H (CDCl₃): 7.4 to 7.15 (ms, 5H); 6.85 (d, 1H, J=7 Hz); 6.8 (ms, 2H); 4.8 (s, 4H); 3.3 to 3.15 (ms, 4H); 2.7 to 2.5 (ms, 4H); 2.5 to 2.3 (ms, 4H); 1.9 to 1.7 (ms, 2H); 1.7 to 1.5 (ms, 2H); 1.5 to 1.35 (ms, 2H)

### Example 5

### Preparation of 1-isoindolin-2-yl-6-[4-(6,7,8,9-tetrahydro-5H-benzocylohepten-2-yl)piperazin-1-yl]hexan-1-one

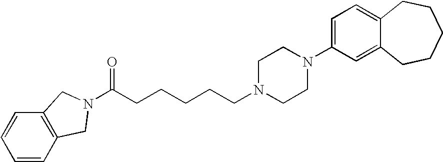

### Step a: Preparation of 6,7,8,9-tetrahydro-5H-benzocyclohepten-2-ylamine

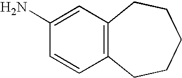

A solution of 1.4 g (7.3 mmol) of 2-nitro-6,7,8,9-tetrahydro-5H-benzo-cycloheptene in 15 mL of methanol containing 0.3 g of 5% palladium on charcoal is hydrogenated for 16 hours at ambient temperature.

After filtration on a bed of celite and concentration of the solvent, 1.25 g of 6,7,8,9-tetrahydro-5H-benzocyclohepten-2-ylamine is obtained.

Rf: 0.77 dichloromethane/methanol/ammonia 90/10/1

NMR ¹H (CDCl₃): 6.9 (d, 1H, J=10 Hz); 6.6 to 6.35 (ms, 2H); 3.7 to 3.25 (s wide, 2H); 2.8 to 2.6 (ms, 4H); 1.9 to 1.75 (ms, 2H); 1.75 to 1.5 (ms, 4H)

2-nitro-6,7,8,9-tetrahydro-5H-benzocycloheptene can be obtained according to the process described in J. Am. Chem. Soc. (1969), 91, 3558-3566.

### Step b: Preparation of 1-(6,7,8,9-tetrahydro-5H-benzocyclohepten-2-yl)-piperazine

A solution of 1.25 g (7.7 mmol) of aniline and 1.4 g (7.7 mmol) of bis(2-chloroethyl)amine hydrochloride in 8 mL of chlorobenzene is brought to reflux for 2 days.

The solution is diluted with diethyl oxide and water. The organic phase is separated out. The aqueous phase is washed again with diethyl oxide then basified with an aqueous solution of soda 10N and extracted twice with diethyl oxide. The ethereal phases are combined, washed with water, dried on magnesium sulphate, filtered and concentrated. In this way 1.42 g of 1-(6,7,8,9-tetrahydro-5H-benzocyclohepten-2-yl)-piperazine is obtained.

Rf: 0.43 dichloromethane/methanol/ammonia 90/10/1

NMR ¹H (CDCl₃): 7.1 (d, 1H, J=10 Hz); 6.7 (d, 1H, J=2.5 Hz); 6.65 (d, 1H, J=10 Hz); 3.2 to 2.9 (ms, 8H); 2.85 (ms, 4H); 1.9 to 1.75 (ms, 2H); 1.75 to 1.5 (ms, 5H)

### Step c: Preparation of ethyl 6-[4-(6,7,8,9-tetrahydro-5H-benzocylohepten-2-yl)piperazin-1-yl]hexanoate

Operating as in Example 1, step a, but starting from 0.8 g of ethyl 6-[4-(6,7,8,9-tetrahydro-5H-benzocylohepten-2-yl)piperazin-1-yl]hexanoate and 0.85 g of ethyl 6-bromohexanoate, 1.15 g of ethyl 6-[4-(6,7,8,9-tetrahydro-5H-benzocylohepten-2-yl)piperazin-1-yl]hexanoate is obtained which are used as such in the subsequent syntheses.

### Step d: Preparation of 6-[4-(6,7,8,9-tetrahydro-5H-benzocylohepten-2-yl)piperazin-1-yl]hexanoic acid

Operating as in Example 1, step b, but starting from 1.1 g of ethyl 6-[4-(6,7,8,9-tetrahydro-5H-benzocylohepten-2-yl)piperazin-1-yl]hexanoate, 0.9 g of 6-[4-(6,7,8,9-tetrahydro-5H-benzocylohepten-2-yl)piperazin-1-yl]hexanoic acid is obtained which is used as such in the subsequent syntheses.

### Step e: 1-isoindolin-2-yl-6-[4-(6,7,8,9-tetrahydro-5H-benzocylohepten-2-yl)piperazin-1-yl]hexan-1-one

Operating as in Example 1, step c, but starting from 0.34 g of 6-[4-(6,7,8,9-tetrahydro-5H-benzocylohepten-2-yl)piperazin-1-yl]hexanoic acid and 0.12 g of isoindoline, 0.3 g of 1-isoindolin-2-yl-6-[4-(6,7,8,9-tetrahydro-5H-benzocylohepten-2-yl)piperazin-1-yl]hexan-1-one is obtained in the form of a solid.

Melting point: 127° C.

NMR ¹H (CDCl₃): 7.4 to 7.2 (ms, 4H); 7.0 (s, 1H, J=7.5 Hz); 6.7 (s, 1H); 6.7 (d, 1H, J=7.5 Hz); 4.8 (s, 4H); 3.25 to 3.1 (ms, 4H); 2.8 to 2.65 (ms, 4H); 2.65 to 2.5 (ms, 4H); 2.5 to 2.3 (ms, 4H); 1.9 to 1.7 (ms, 4H); 1.7 to 1.5 (ms, 6H); 1.5 to 1.35 (ms, 2H)

### Example 6

### Preparation of 1-(4-fluoroisoindolin-2-yl)-6-[4-(3-trifluoromethylphenyl)piperazin-1-yl]hexan-1-one

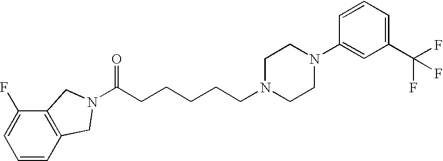

Operating as in Example 1, step c, but starting from 0.14 g of 4-fluoro-isoindoline (preparation described in EP 0 343 560 A2) and 0.34 g of 6-[4-(3-trifluoromethoxyphenyl)piperazin-1-yl]hexanoic acid, 0.11 g of 1-(4-fluoroisoindolin-2-yl)-6-[4-(3-trifluoromethylphenyl)piperazin-1-yl]-hexan-1-one in the form of a white solid.

NMR ¹H (CDCl₃): 7.4 to 7.25 (ms, 2H); 7.2 to 6.9 (ms, 5H); 4.8 (s, 4H); 3.25 (m, 4H); 2.6 (m, 4H); 2.6 to 2.3 (ms, 4H); 1.8 (m, 2H); 1.7 to 1.2 (ms, 4H)

### Example 7

### Preparation of 1-(5-fluoroisoindolin-2-yl)-6-[4-(3-trifluoromethylphenyl)piperazin-1-yl]hexan-1-one

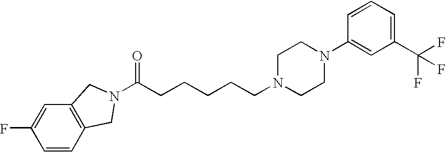

Operating as in Example 1, step c, but starting from 0.14 g 5-fluoro-isoindoline (preparation described in EP0343 560 A2) and 0.34 g 6-[4-(3-trifluoromethoxyphenyl)piperazin-1-yl]hexanoic acid, 0.11 g 1-(5-fluoroisoindolin-2-yl)-6-[4-(3-trifluoromethylphenyl)piperazin-1-yl]hexan-1-one is obtained in the form of a beige solid.

NMR ¹H (CDCl₃): 7.4 (m, 1H); 7.25 (m, 1H); 7.15 to 6.9 (ms, 5H); 4.8 (s, 4H); 3.2 (m, 4H); 2.65 (m, 4H); 2.5 (m, 2H); 2.4 (t, 2H, J=7 Hz); 1.8 (m, 2H); 1.75 to 1.35 (ms, 4H)

### Example 8

### Preparation of 1-(5-isopropoxyisoindolin-2-yl)-6-[4-(3-trifluoromethylphenyl)piperazin-1-yl]hexan-1-one

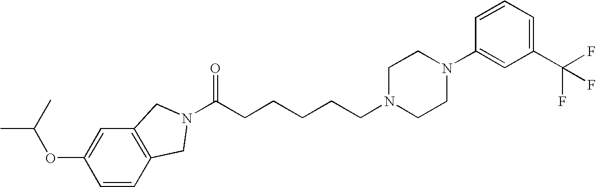

### Step a: Preparation of 1-(5-hydroxyisoindolin-2-yl)-6-[4-(3-trifluoromethylphenyl)piperazin-1-yl]hexan-1-one

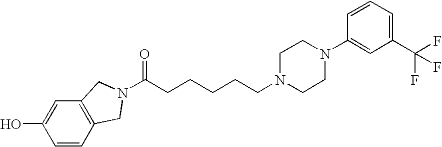

Operating as in Example 1, step c, but starting from 0.47 g of 5-hydroxyisoindoline (preparation described in EP 0 343 560 A2) and 0.76 g of 6-[4-(3-trifluoromethoxyphenyl)piperazin-1-yl]hexanoic acid, 0.7 g of 1-(5-hydroxyisoindolin-2-yl)-6-[4-(3-trifluoromethylphenyl)piperazin-1-yl]hexan-1-one is obtained in the form of oil.

### Step b: Preparation of 1-(5-isopropoxyisoindolin-2-yl)-6-[4-(3-trifluoromethylphenyl)piperazin-1-yl]hexan-1-one

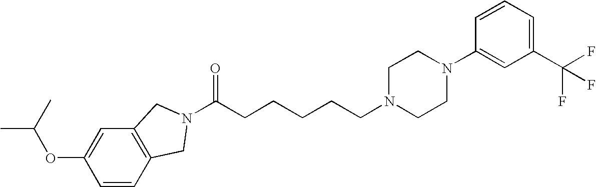

A mixture of 0.17 g (0.37 mmol) of 1-(5-hydroxyisoindolin-2-yl)-6-[4-(3-trifluoromethylphenyl)piperazin-1-yl]hexan-1-one, 0.18 g (0.55 mmol) of caesium carbonate, 75 mg of 2-iodopropane and 5 mL of acetonitrile is brought to reflux for 16 hours.

After concentration of the solvent, the residue is taken up with ethyl acetate and washed with water. The organic phase is dried on magnesium sulphate, filtered and concentrated. The product is purified by silica gel chromatography (dichloromethane/methanol eluent 95/5). In this way 38 mg of 1-(5-isopropoxyisoindolin-2-yl)-6-[4-(3-trifluoromethylphenyl)piperazin-1-yl]hexan-1-one is obtained in the form of beige solid.

NMR ¹H (CDCl₃): 7.4 (m, 1H); 7.2 to 70 (ms, 4H); 6.9 to 6.7 (ms, 2H); 4.75 (s, 2H); 4.7 (s, 2H); 4.55 (m, 1H); 3.2 (m, 4H); 2.6 (m, 4H); 2.55 to 2.2 (ms, 4H); 1.8 (m, 2H); 1.75 to 1.35 (ms, 4H); 1.35 (d, 6H, j=7 Hz)

### Example 9

### Preparation of 1-(4-chloroisoindolin-2-yl)-6-[4-(3-trifluoromethylphenyl)piperazin-1-yl]hexan-1-one

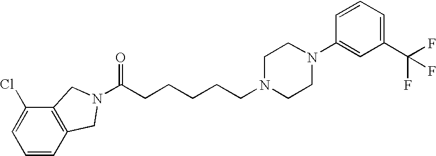

Operating as in Example 1, step c, but starting from 0.16 g of 4-chloroisoindoline (preparation described in EP 0 343 560 A2) and 0.34 g of 6-[4-(3-trifluoromethoxyphenyl)piperazin-1-yl]hexanoic acid, 0.30 g of 1-(4-chloroisoindolin-2-yl)-6-[4-(3-trifluoromethylphenyl)piperazin-1-yl]hexan-1-one is obtained in the form of a white solid.

NMR ¹H (CDCl₃): 7.4 (m, 1H); 7.3 to 7.0 (ms, 6H); 4.9 (s, 2H); 4.8 (s, 2H); 3.3 (m, 4H); 2.6 (m, 4H); 2.55 to 2.3 (ms, 4H); 1.8 (m, 2H); 1.7 to 1.35 (ms, 4H)

### Example 10

### Preparation of 1-(6,7-dihydro-5H-[1,3]dioxolo[4,5-f]isoindol-6-yl)-6-[4-(3-trifluoromethylphenyl)piperazin-1-yl]hexan-1-one

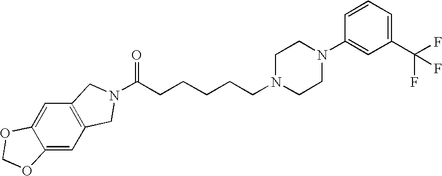

Operating as in Example 1, step c, but starting from 0.16 g 6,7-dihydro-5H-[1,3]dioxolo[4,5-f]isoindoline and 0.34 g 6-[4-(3-trifluoromethoxy-phenyl)piperazin-1-yl]hexanoic acid, 0.22 g 1-(6,7-dihydro-5H-[1,3]dioxolo[4,5-f]isoindol-6-yl)-6-[4-(3-trifluoromethylphenyl)piperazin-1-yl]hexan-1-one is obtained in the form of a brown solid.

NMR ¹H(CDCl₃): 7.35 (m, 1H) 7.2 to 7.0 (ms, 3H); 6.75 (s, 1H); 6.7 (s, 1H); 6.0 (s, 2H); 4.7 (s, 4H); 3.25 (m, 4H); 2.65 (m, 4H); 2.5 to 2.3 (ms, 4H); 1.8 (m, 2H); 1.7 to 1.5 (ms, 2H); 1.5 to 1.3 (ms, 2H)

### Example 11

### Preparation of 1-(5-hydroxymethylisoindolin-2-yl)-6-[4-(3-trifluoromethylphenyl)piperazin-1-yl]hexan-1-one

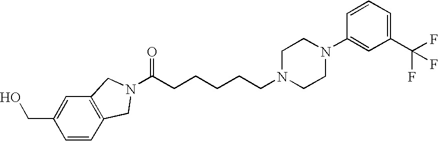

Operating as in Example 1, step c, but starting from 0.05 g of 5-hydroxymethylisoindoline (preparation described in EP 0 343 560 A2) and 0.14 of 6-[4-(3-trifluoromethoxyphenyl)piperazin-1-yl]hexanoic acid, 0.07 g of 1-(5-hydroxymethylisoindolin-2-yl)-6-[4-(3-trifluoromethyl-phenyl)-piperazin-1-yl]hexan-1-one is obtained in the form of a white solid.

NMR ¹H (CDCl₃): 7.4 to 7.2 (ms, 4H); 7.15 to 7.0 (ms, 3H); 4.8 (s, 4H); 4.7 (d, 2H, J=5 Hz); 3.2 (m, 4H); 2.7 (m, 4H); 2.55 to 2.3 (ms, 4H); 1.8 (m, 2H); 1.7 to 1.5 (ms, 3H); 1.45 (m, 2H)

### Example 12

### Preparation of 1-(5-methoxycarbonylisoindolin-2-yl)-6-[4-(3-trifluoromethylphenyl)piperazin-1-yl]hexan-1-one hydrochloride

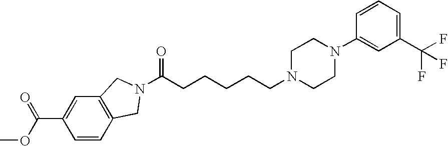

Operating as in Example 1, step c, but starting from 0.60 g of 5-methoxycarbonylisoindoline (preparation described in EP 0 343 560 A2) and 0.90 g of 6-[4-(3-trifluoromethoxyphenyl)piperazin-1-yl]hexanoic acid, 0.74 g of 1-(5-methoxycarbonylisoindolin-2-yl)-6-[4-(3-trifluoromethylphenyl)piperazin-1-yl]hexan-1-one hydrochloride is obtained in the form of a brown solid.

NMR ¹H(CDCl₃): 8.0 (m, 2H); 7.45 to 7.25 (ms, 2H); 7.15 to 7.0 (ms, 3H); 4.8 (s, 4H); 3.9 (s, 3H); 3.2 (m, 4H); 2.65 (m, 4H); 2.55 to 2.3 (ms, 4H); 1.8 (m, 2H); 1.6 (m, 2H); 1.45 (m, 2H)

### Example 13

### Preparation of 1-(5-carboxyisoindolin-2-yl)-6-[4-(3-trifluoromethylphenyl)piperazin-1-yl]hexan-1-one

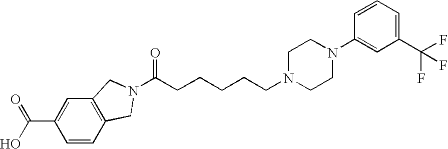

A mixture of 0.61 g of 1-(5-methoxycarbonylisoindolin-2-yl)-6-[4-(3-trifluoromethylphenyl)piperazin-1-yl]hexan-1-one hydrochloride, 10 mL of methanol and 1.5 mL of an aqueous solution of normal soda is brought to reflux for seven hours.

After concentration of the methanol, the residue is taken up with water then acidified with an aqueous solution of normal hydrochloric acid. The precipitate is filtered, washed with diisopropyl oxide and dried in a vacuum on phosphorus pentoxide. In this way 0.48 g (81%) of 1-(5-carboxyisoindolin-2-yl)-6-[4-(3-trifluoromethylphenyl)piperazin-1-yl]hexan-1-one is obtained.

NMR ¹H (DMSO D₆): 7.9 (m, 2H); 7.5 to 7.3 (ms, 2H); 7.2 (ms, 1H); 7.1 (s, 1H); 7.0 (m, 1H); 4.85 (s, 2H); 4.65 (s, 2H); 3.2 (m, 4H); 2.55 (m, 4H); 2.45 to 2.2 (ms, 4H); 1.7 to 1.4 (ms, 4H); 1.3 (m, 2H)

### Example 14

### Preparation of 1-[5-(2-hydroxyethylaminocarbonyl)isoindolin-2-yl]-6-[4-(3-trifluoromethylphenyl)piperazin-1-yl]hexan-1-one

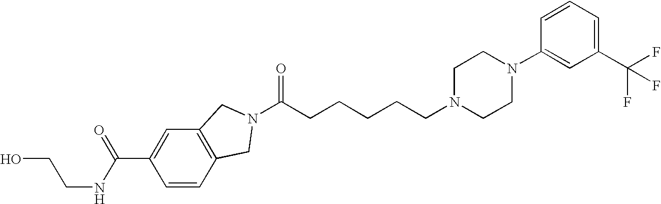

Operating as in Example 1, step c, but starting from 0.11 g of 1-(5-carboxyisoindolin-2-yl)-6-[4-(3-trifluoromethylphenyl)piperazin-1-yl]hexan-1-one and 14 mg of ethanolamine, 6 mg of 1-[5-(2-hydroxyethylamino-carbonyl)isoindolin-2-yl]-6-[4-(3-trifluoromethylphenyl)piperazin-1-yl]hexan-1-one is obtained in the form of a white solid.

NMR ¹H (CDCl₃): 7.8 to 7.6 (ms, 2H); 7.4 (m, 2H); 7.15 to 7.0 (ms, 3H); 6.6 (d wide, 1H); 4.8 (s, 4H); 3.85 (m, 2H); 3.75 (m, 2H); 3.2 (m, 4H); 2.6 (m, 4H); 2.5 to 2.3 (ms, 4H); 1.8 (m, 2H); 1.7 to 1.35 (ms, 4H)

### Example 15

### Preparation of 1-(5-morpholinocarbonylisoindolin-2-yl)-6-[4-(3-trifluoromethylphenyl)piperazin-1-yl]hexan-1-one

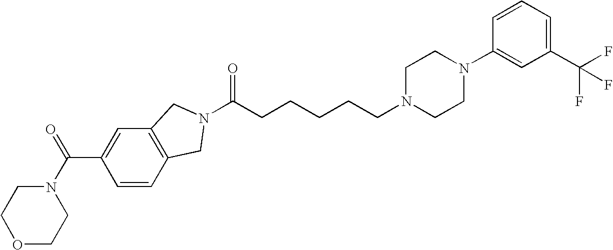

Operating as in Example 1, step c, but starting from 0.10 g of 1-(5-carboxyisoindolin-2-yl)-6-[4-(3-trifluoromethylphenyl)piperazin-1-yl]hexan-1-one and 20 mg of morpholine, 51 mg of 1-(5-morpholinocarbonyl-isoindolin-2-yl)-6-[4-(3-trifluoromethylphenyl)piperazin-1-yl]hexan-1-one is obtained in the form of a yellow solid.

NMR ¹H (CDCl₃): 7.45 to 7.3 (ms, 4H); 7.2 to 7.0 (ms, 3H); 4.8 (s, 4H); 3.9 to 3.5 (ms, 8H); 3.3 (m, 4H); 2.65 (m, 4H); 2.55 to 2.3 (ms, 4H); 1.8 (m, 2H); 1.7 to 1.4 (ms, 4H)

### Example 16

### Preparation of 1-(5-(pyrrolidin-1-ylcarbonylisoindolin-2-yl)-6-[4-(3-trifluoromethylphenyl)piperazin-1-yl]hexan-1-one

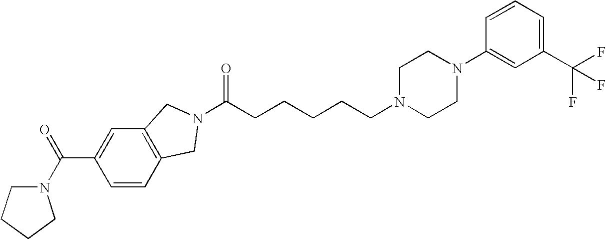

Operating as in Example 1, step c, but starting from 0.10 g of 1-(5-carboxyisoindolin-2-yl)-6-[4-(3-trifluoromethylphenyl)piperazin-1-yl]hexan-1-one and 14 mg of pyrrolidine, 38 mg of 1-(5-(pyrrolidin-1-ylcarbonylisoindolin-2-yl)-6-[4-(3-trifluoromethylphenyl)piperazin-1-yl]hexan-1-one is obtained in the form of a yellow solid.

Melting point: 109° C.

NMR ¹H (CDCl₃): 7.5 to 7.4 (ms, 2H); 7.4 to 7.2 (ms, 2H); 7.2 to 7.0 (ms, 3H); 4.8 (s, 4H); 3.65 (m, 2H); 3.45 (m, 2H); 3.35 (m, 4H); 2.7 (m, 4H); 2.6 (m, 2H); 2.4 (t, 2H, J=7 Hz); 2.1 to 1.4 (ms, 10H)

### Example 17

### Preparation of 1-(5-piperidin-1-ylcarbonylisoindolin-2-yl)-6-[4-(3-trifluoromethylphenyl)piperazin-1-yl]hexan-1-one

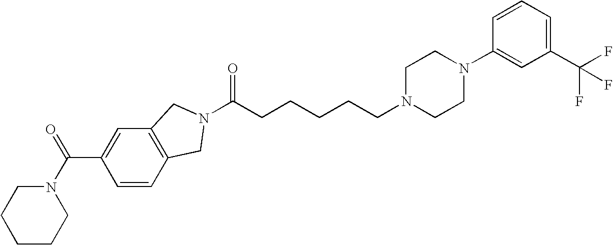

Operating as in Example 1, step c, but starting from 0.10 g of 1-(5-carboxyisoindolin-2-yl)-6-[4-(3-trifluoromethylphenyl)piperazin-1-yl]hexan-1-one and 17 mg of piperidine, 42 mg of 1-(5-piperidin-1-ylcarbonylisoindolin-2-yl)-6-[4-(3-trifluoromethylphenyl)piperazin-1-yl]hexan-1-one is obtained in the form of a yellow solid.

Melting point: 91° C.

NMR ¹H (CDCl₃): 7.5 to 7.25 (ms, 4H); 7.2 to 7.0 (ms, 3H); 4.8 (s, 4H); 3.7 (m, 2H); 3.5 to 3.2 (ms, 6H); 2.9 to 2.5 (ms, 6H); 2.4 (t, 2H, J=7 Hz); 1.9 to 1.4 (ms, 12H)

### Example 18

### Preparation of 1-[5-(2-dimethylamino-ethylamino-carbonyl)-isoindolin-2-yl]-6-[4-(3-trifluoromethylphenyl)piperazin-1-yl]hexan-1-one

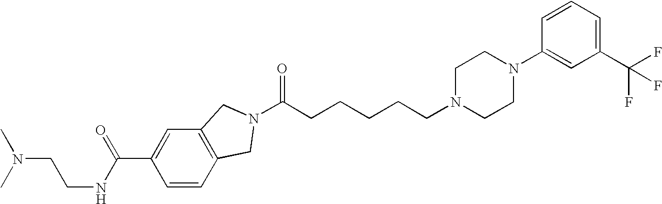

Operating as in Example 1, step c, but starting from 0.10 g of 1-(5-carboxyisoindolin-2-yl)-6-[4-(3-trifluoromethylphenyl)piperazin-1-yl]hexan-1-one and 12 mg of 2-dimethylaminoethylamine, 49 mg of 1-[5-(2-dimethylaminoethylaminocarbonyl)isoindolin-2-yl]-6-[4-(3-trifluoromethylphenyl)piperazin-1-yl]hexan-1-one is obtained in the form of a white solid.

Melting point: 120° C.

NMR ¹H (CDCl₃): 7.9 to 7.75 (ms, 2H); 7.35 (m, 2H); 7.15 to 7.0 (ms, 3H); 4.85 (s, 4H) 3.65 (m, 2H); 3.3 (m, 4H); 2.8 (m, 2H); 2.65 (m, 4H); 2.55 to 2.3 (ms, 10H); 1.8 (m, 2H) 1.6 (m, 2H); 1.45 (m, 2H)

### Example 19

### Preparation of 1-[5-(4,5-dihydrooxazol-2yl)isoindolin-2-yl]-6-[4-(3-trifluoromethylphenyl)piperazin-1-yl]hexan-1-one

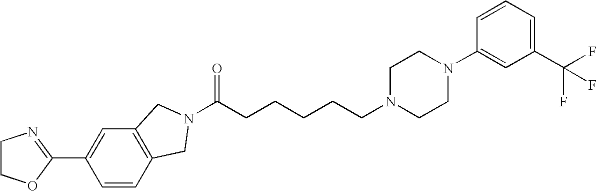

### Step a: Preparation of 1-[5-(2-chloroethylaminocarbonyl)isoindolin-2-yl]-6-[4-(3-trifluoromethylphenyl)piperazin-1-yl]hexan-1-one

Operating as in Example 1, step c, but starting from 0.20 g of 1-(5-carboxyisoindolin-2-yl)-6-[4-(3-trifluoromethylphenyl)piperazin-1-yl]hexan-1-one and 52 mg of 2-chloroethylamine hydrochloride, 0.19 g of 1-[5-(2-chloroethylaminocarbonyl)isoindolin-2-yl]-6-[4-(3-trifluoromethylphenyl)piperazin-1-yl]hexan-1-one is obtained in the form of a solid.

NMR ¹H (CDCl₃): 7.9 (m, 1H); 7.7 (m, 1H); 7.5 to 7.2 (ms, 2H); 7.2 to 7.0 (ms, 3H); 6.6 (m, 1H); 4.8 (s, 4H); 4.45 (t, 2H, J=7 Hz); 4.1 (m, 2H); 3.4 (m, 4H); 3.0 (m, 4H); 2.8 (m, 2H); 2.4 (m, 2H); 1.9 to 1.75 (ms, 4H); 1.45 (m, 2H)

### Step b: Preparation of 1-[5-(4,5-dihydrooxazol-2yl)isoindolin-2-yl]-6-[4-(3-trifluoromethylphenyl)piperazin-1-yl]hexan-1-one

A solution of 0.18 g of 1-[5-(2-chloroethylaminocarbonyl)isoindolin-2-yl]-6-[4-(3-trifluoromethylphenyl)piperazin-1-yl]hexan-1-one and 0.11 g of 1.8-diazabicyclo[5.4.0]undec-7-ene in 5 mL of dichloromethane is heated to reflux during one night.

After concentration of the solvent, the residue is diluted with water. The solid is filtered then purified by silica gel chromatography (eluent dichloro-methane/methanol 95/5). In this way 45 mg of 1-[5-(4,5-dihydro-oxazol-2yl)isoindolin-2-yl]-6-[4-(3-trifluoromethylphenyl)piperazin-1-yl]hexan-1-one is obtained in the form of a white solid.

Melting point: 129° C.

NMR ¹H (CDCl₃): 7.9 (m, 2H); 7.45 to 7.2 (ms, 3H); 7.2 to 7.0 (ms, 4H); 4.8 (s, 4H); 4.5 (t, 2H, J=7 Hz); 4.1 (t, 2H, J=7 Hz); 3.45 (m, 4H); 3.0 to 2.5 (ms, 6H); 2.4 (t, 2H, J=7 Hz); 1.8 (m, 2H); 1.7 to 1.4 (ms, 4H)

### Example 20

### Preparation of 1-(5-morpholinoylmethyl-isoindolin-2-yl)-6-[4-(3-trifluoromethylphenyl)piperazin-1-yl]hexan-1-one hydrochloride

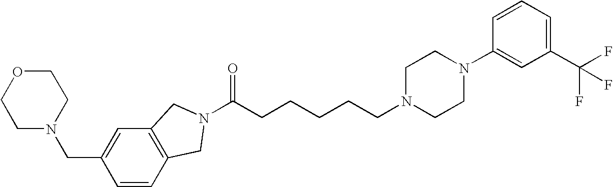

### Step a: Preparation of 1-(5-methanesulphonyloxymethylisoindolin-2-yl)-6-[4-(3-trifluoromethylphenyl)piperazin-1-yl]hexan-1-one

A solution of 0.17 g (0.36 mmol) of 1-(5-hydroxymethylisoindolin-2-yl)-6-[4-(3-trifluoromethylphenyl)piperazin-1-yl]hexan-1-one and 40 mg (0.40 mmol) of triethylamine in 2.5 mL of dichloromethane is cooled to −15° C. 45 mg (0.40 mmol) of methanesulphonyl chloride are then added.

The mixture is stirred for four hours at ambient temperature then is again cooled to −15° C. 23 mg of methanesulphonyl chloride are added. Stirring is continued for one hour at ambient temperature.

The medium is diluted with dichloromethane and washed with water. The organic phase is dried on magnesium sulphate, filtered and concentrated. In this way 0.18 g of 1-(5-methanesulphonyloxymethylisoindolin-2-yl)-6-[4-(3-trifluoro-methylphenyl)piperazin-1-yl]hexan-1-one is obtained which is used in crude form in the following step.

### Step b: Preparation of 1-(5-morpholinomethylisoindolin-2-yl)-6-[4-(3-trifluoromethylphenyl)piperazin-1-yl]hexan-1-one

A mixture of 0.18 g (0.32 mmol) of 1-(5-methanesulphonyloxymethylisoindolin-2-yl)-6-[4-(3-trifluoromethylphenyl)piperazin-1-yl]hexan-1-one, 28 mg (0.32 mmol) of morpholine, 49 mg (0.35 mmol) of potassium carbonate and 5 mL of acetonitrile is brought to reflux during one night.

After concentration of the solvent, the residue is taken up with ethyl acetate then washed with water. The organic phase is dried on magnesium sulphate, filtered and concentrated. The product is purified by silica gel chromatography (eluent dichloromethane/methanol 95/5) then salified by a saturated solution of hydrochloric acid in diethyl oxide. In this way 47 mg of 5% 1-(5-morpholinomethylisoindolin-2-yl)-6-[4-(3-trifluoromethylphenyl)piperazin-1-yl]hexan-1-one hydrochloride is obtained in the form of a hygroscopic beige solid.

NMR ¹H (DMSO D₆): 9.9 (s, wide, 1H); 7.4 (m, 1H); 7.4 to 7.0 (ms, 6H); 4.8 (s, 2H); 4.6 (s, 2H); 4.0 (m, 2H); 3.7 to 3.3 (ms, 8H); 3.25 to 3.0 (ms, 8H); 2.45 to 2.2 (ms, 4H); 1.8 to 1.45 (ms, 4H); 1.35 (m, 2H)

### Example 21

### Preparation of 1-[5-(pyrrolidin-1-ylmethyl)isoindolin-2-yl]-6-[4-(3-trifluoromethylphenyl)piperazin-1-yl]hexan-1-one

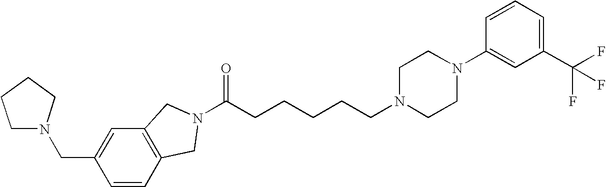

Operating as in Example 20, step b, but starting from 0.18 g of 1-(5-methanesulphonyloxymethylisoindolin-2-yl)-6-[4-(3-trifluoromethylphenyl)piperazin-1-yl]hexan-1-one and 27 mg of pyrrolidine, 37 mg of 1-[5-(pyrrolidin-1-ylmethyl)isoindolin-2-yl]-6-[4-(3-trifluoromethylphenyl)piperazin-1-yl]hexan-1-one is obtained in the form of a yellow solid.

NMR ¹H (CDCl₃): 7.4 to 7.15 (ms, 4H); 7.15 to 7.0 (ms, 3H); 4.8 (s, 4H); 3.75 (m, 2H); 3.25 (m, 4H); 2.8 to 2.5 (ms, 8H); 2.5 to 2.3 (ms, 4H); 1.9 (m, 4H); 1.8 (m, 2H); 1.6 (m, 2H); 1.45 (m, 2H)

### Example 22

### Preparation of 1-[5-(dimethylaminomethyl)isoindolin-2-yl]-6-[4-(3-trifluoromethylphenyl)piperazin-1-yl]hexan-1-one

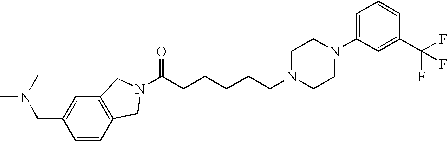

Operating as in Example 20, step b, but starting from 0.29 g of 1-(5-methanesulphonyloxymethylisoindolin-2-yl)-6-[4-(3-trifluoromethylphenyl)piperazin-1-yl]hexan-1-one and 0.8 mL of a solution of dimethylamine in 2M tetrahydrofuran, 55 mg of 1-[5-(dimethylaminomethyl)isoindolin-2-yl]-6-[4-(3-trifluoromethylphenyl)piperazin-1-yl]hexan-1-one is obtained in the form of a beige solid.

Melting point: 77° C.

NMR ¹H (CDCl₃): 7.45 to 7.2 (ms, 4H); 7.2 to 7.0 (ms, 3H); 4.8 (s, 4H); 3.55 (m, 2H); 3.25 (m, 4H); 2.65 (m, 4H); 2.55 to 2.2 (ms, 10H); 1.8 (m, 2H); 1.6 (m, 2H); 1.45 (m, 2H)

### Example 23

### Preparation of 1-[5-(2-piperidinoethoxy)isoindolin-2-yl]-6-[4-(3-trifluoromethylphenyl)piperazin-1-yl]hexan-1-one

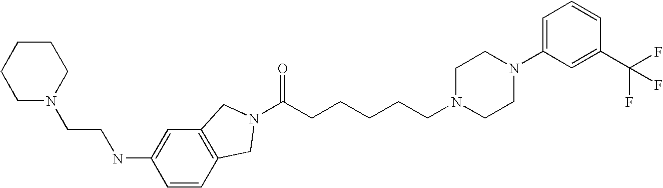

A mixture of 0.1 g (0.22 mmol) of 1-(5-hydroxyisoindolin-2-yl)-6-[4-(3-trifluoromethylphenyl)piperazin-1-yl]hexan-1-one (Example 8, step a), 40 mg (0.22 mmol) of 1-(2-chloroethyl)piperidine hydrochloride, 64 mg (0.46 mmol) of potassium carbonate and 8 mL of acetonitrile is brought to reflux during one night.

After concentration of the solvent, the residue is taken up with ethyl acetate then is washed with water. The organic phase is dried on magnesium sulphate, filtered and concentrated. The residue is crystallised in pentane, filtered and dried in a vacuum. In this way 64 mg of 1-[5-(2-piperidinoethoxy)isoindolin-2-yl)-6-[4-(3-trifluoromethylphenyl)piperazin-1-yl]hexan-1-one is obtained in the form of a white solid.

Melting point: 88° C.

NMR ¹H (CDCl₃): 7.4 (m, 1H); 7.2 to 7.0 (ms, 4H); 6.8 (m, 2H); 4.75 (s, 4H); 4.25 (m, 2H); 3.25 (m, 4H); 3.0 (m, 2H); 2.8 to 2.5 (ms, 6H); 2.5 to 2.3 (ms, 4H); 1.8 (m, 2H); 1.7 to 1.3 (ms, 12H)

### Example 24

### Preparation of 1-[5-(3-piperidinopropoxy)isoindolin-2-yl]-6-[4-(3-trifluoromethylphenyl)piperazin-1-yl]hexan-1-one

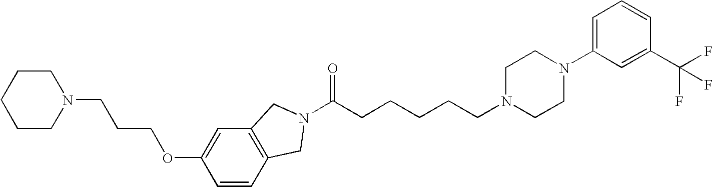

Operating as in Example 23, but starting from 0.10 g of 1-(5-hydroxyisoindolin-2-yl)-6-[4-(3-trifluoromethylphenyl)piperazin-1-yl]hexan-1-one (Example 8, step a) and 44 mg of 1-(3-chloropropylpiperidine hydrochloride, 75 mg of 1-[5-(3-piperidinopropoxy)isoindolin-2-yl]-6-[4-(3-trifluoro-methylphenyl)piperazin-1-yl]hexan-1-one is obtained in the form of a beige solid.

Melting point: 92° C.

NMR ¹H (CDCl₃): 7.35 (m, 1H); 7.25 to 7.0 (ms, 4H); 6.9 to 6.75 (ms, 2H); 4.75 (s, 2H); 4.7 (s, 2H); 4.0 (t, 2H, J=7 Hz); 3.25 (m, 4H); 2.75 to 2.45 (ms, 8H); 2.45 to 2.3 (ms, 6H); 2.1 (m, 2H); 1.9 to 1.35 (ms, 12H)

### Example 25

### Preparation of 1-[5-(3-pyrrolidinopropoxy)isoindolin-2-yl]-6-[4-(3-trifluoromethylphenyl)piperazin-1-yl]hexan-1-one

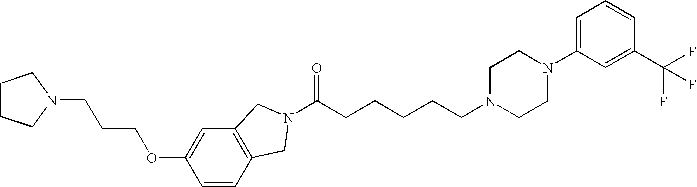

Operating as in Example 23, but starting from 0.10 g of 1-(5-hydroxyisoindolin-2-yl)-6-[4-(3-trifluoromethylphenyl)piperazin-1-yl]hexan-1-one (Example, step a) and 40 mg of 1-chloropropylpyrrolidine hydrochloride, 30 mg of 1-[5-(3-pyrrolidinopropoxy)isoindolin-2-yl]-6-[4-(3-trifluoro-methylphenyl)piperazin-1-yl]hexan-1-one is obtained in the form of a beige solid.

Melting point: 92° C.

NMR ¹H (CDCl₃): 7.35 (m, 1H); 7.25 to 7.0 (ms, 4H); 6.9 to 6.75 (ms, 2H); 4.75 (s, 4H); 4.05 (t, 2H, J=7 Hz); 3.25 (m, 4H); 2.95 to 2.65 (ms, 6H); 2.6 (m, 4H); 2.5 to 2.3 (ms, 4H); 2.15 (m, 2H); 1.9 (m, 2H); 1.8 (m, 2H); 1.6 (m, 2H); 1.45 (m, 2H)

### Example 26

### Preparation of 1-(5-acetylisoindolin-2-yl)-6-[4-(3-trifluoromethylphenyl)piperazin-1-yl]hexan-1-one

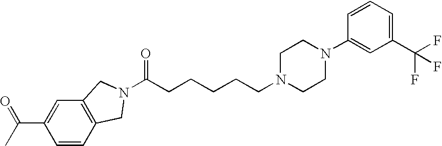

### Step a: Preparation of 5-acetylisoindoline

A solution of 0.3 g (1.7 mmol) of 5-methoxycarbonylisoindoline in 4 mL of tetrahydrofuran is heated to 50° C. A solution of 3.3 mL (8.5 mmol) of methylmagnesium chloride in tetrahydrofuran is then added drop by drop.

The reaction medium is brought to reflux for three hours then is cooled to 0° C. 40 mL of water are added. The suspension is filtered on celite. The filtrate is extracted twice with dichloromethane. The extraction phases are combined, dried on magnesium sulphate, filtered and concentrated. In this way 0.24 g of 5-acetylisoindoline is obtained.

### Step b: Preparation of 1-(5-acetylisoindolin-2-yl)-6-[4-(3-trifluoromethylphenyl)piperazin-1-yl]hexan-1-one

Operating as in Example 1, step c, but starting from 0.23 g of 5-acetylisoindoline and 0.50 g of 6-[4-(3-trifluoromethoxyphenyl)piperazin-1-yl]hexanoic acid, 45 mg of 1-(5-acetylisoindolin-2-yl)-6-[4-(3-trifluoro-methylphenyl)piperazin-1-yl]hexan-1-one is obtained in the form of a brown solid.

NMR ¹H (CDCl₃): 8.0 to 7.9 (ms, 2H); 7.45 to 7.25 (ms, 2H); 7.15 to 7.0 (ms, 3H); 4.85 (s, 4H); 3.3 (m, 4H); 2.7 (m, 4H); 2.65 (s, 3H); 2.55 to 2.35 (ms, 4H); 1.8 (m, 2H); 1.65 (m, 2H); 1.45 (m, 2H)

### Example 27

### Preparation of 1-[5-(1-hydroxy-1-methylethyl)isoindolin-2-yl]-6-[4-(3-trifluoromethylphenyl)piperazin-1-yl]hexan-1-one

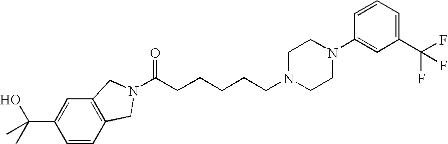

0.67 mL (1.75 mmol) of a solution of methylmagnesium chloride in tetrahydrofuran are added to a solution of 0.25 g (0.5 mmol) of 1-(5-methoxycarbonylisoindolin-2-yl)-6-[4-(3-trifluoromethylphenyl)piperazin-1-yl]hexan-1-one in 2 mL of tetrahydrofuran cooled to −2° C. The mixture is stirred for two hours at ambient temperature then is cooled to −2° C. 0.38 mL (1 mmol) of a solution of methylmagnesium chloride in tetrahydrofuran is added. The mixture is stirred for one hour at ambient temperature then is cooled to 0° C.

Water is added then the medium is diluted with ethyl acetate. The suspension is filtered on celite. The organic phase is decanted, dried on magnesium sulphate, filtered and concentrated. The product is purified by silica gel chromatography (eluent dichloromethane/methanol 95/5). In this way 38 mg (15%) of 1-[5-(1-hydroxy-1-methylethyl)isoindolin-2-yl]-6-[4-(3-trifluoromethylphenyl)piperazin-1-yl]hexan-1-one is obtained in the form of oil.

NMR ¹H (CDCl₃): 7.5 to 7.2 (ms, 4H); 7.2 to 7.0 (ms, 3H); 4.8 (s, 4H); 3.2 (m, 4H); 2.65 (m, 4H); 2.55 to 2.3 (ms, 4H); 1.8 (m, 2H); 1.75 to 1.55 (ms, 9H); 1.45 (m, 2H)

### Example 28

### Preparation of 1-[5-(1-hydroxyethyl)isoindolin-2-yl]-6-[4-(3-trifluoromethylphenyl)piperazin-1-yl]hexan-1-one hydrochloride

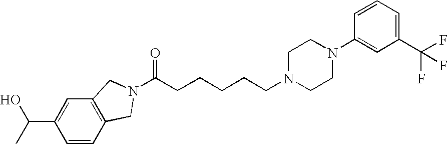

8 mg (0.2 mmol) of sodium borohydride at 5° C. are added to a solution of 99 mg (0.2 mmol) of 1-(5-acetylisoindolin-2-yl)-6-[4-(3-trifluoromethylphenyl)piperazin-1-yl]hexan-1-one in 4 mL of methanol. The solution is stirred for two hours at ambient temperature. After concentration of the methanol, the residue is extracted with ethyl acetate and washed with water. The organic phase is dried on magnesium sulphate, filtered and concentrated.

The oily residue is dissolved in diethyl oxide and acidified with a saturated solution of hydrogen chloride in diethyl oxide. The salt is filtered, washed with diethyl oxide and dried in a vacuum. In this way 33 mg of 1-[5-(1-hydroxyethyl)isoindolin-2-yl]-6-[4-(3-trifluoro-methylphenyl)piperazin-1-yl]hexan-1-one hydrochloride is obtained.

NMR ¹H (DMSO D₆): 10.2 (s wide, 1H); 7.45 (m, 1H); 7.35 to 7.15 (ms, 5H); 7.1 (m, 1H); 4.8 (s, 2H); 4.7 (m, 1H); 4.6 (s, 2H); 4.0 (m, 2H); 3.6 to 3.3 (ms, 4H); 3.2 to 3.0 (ms, 4H); 2.3 (m, 2H); 1.75 (m, 2H); 1.6 (m, 2H); 1.4 (m, 2H); 1.3 (d, 3H, J=7 Hz)

### Example 29

### Preparation of 1-(5-aminocarbonylisoindolin-2-yl)-6-[4-(3-trifluoromethylphenyl)piperazin-1-yl]hexan-1-one

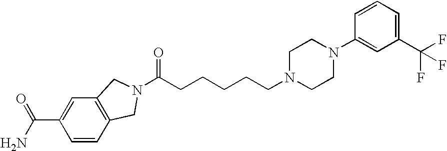

A mixture of 0.14 g (0.28 mmol) of 1-(5-carboxyisoindolin-2-yl)-6-[4-(3-trifluoromethylphenyl)piperazin-1-yl]hexan-1-one (Example 13), 38 mg (0.28 mmol) of hydroxybenzotriazole, 54 mg (0.28 mmol) of 1-(3-dimethylaminopropyl)-3-ethylcarbodiimide hydrochloride, 30 mg (0.56 mmol) of ammonium chloride and 5 mL of tetrahydrofuran is cooled to 0° C. A solution of 85 mg (0.84 mmol) of triethylamine in 0.5 mL of dichloromethane is added.

The mixture is stirred at ambient temperature during one night. The solvent is concentrated in a vacuum and the residue is diluted with water. The solid is filtered and dried in a vacuum. In this way 78 mg (57%) of 1-(5-aminocarbonylisoindolin-2-yl)-6-[4-(3-trifluoromethylphenyl)piperazin-1-yl]hexan-1-one is obtained in the form of a beige solid.

NMR ¹H (CDCl₃): 7.85 to 7.65 (ms, 2H); 7.4 (m, 2H); 7.2 to 7.0 (ms, 3H); 6.1 (s wide, 1H); 5.7 (s wide, 1H); 4.85 (s, 4H); 3.35 (m, 4H); 2.75 (m, 4H); 2.55 (m, 2H); 2.4 (t, 2H, J=7 Hz); 1.9 to 1.6 (ms, 4H); 1.5 (m, 2H)

### Example 30

### Preparation of 1-isoindolin-2-yl-6-[4-(3-trifluoromethylphenyl)piperazin-1-yl]hexan-1-one hydrochloride

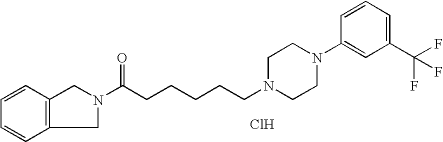

### Step a: Preparation of 2-(6-bromohexanoyl)isoindoline

A solution containing 1.2 g (10 mmol) of isoindoline, 20 mL of dichloromethane and 1.5 mL (11 mmol) of triethylamine is cooled to 5° C. 1.53 mL (10 mmol) of 6-bromohexanoyl chloride is then added at 5° C. The cooling bath is removed and stirring is continued for 6 hours at ambient temperature.

The reaction medium is diluted with 30 mL of dichloromethane and is washed with water (twice 50 mL). The organic phase is dried on magnesium sulphate, filtered and concentrated in a vacuum. The crystallised residue obtained is stirred with 10 mL of diisopropyl oxide. After filtration and drying in a vacuum, 2.65 g of 2-(6-bromohexanoyl)isoindoline is obtained in the form of white crystals.

Melting point: 88° C.

NMR ¹H (CDCl₃): 7.45 to 7.2 (ms, 4H); 4.8 (s, 4H); 3.45 (t, 2H, J=7.5 Hz); 2.4 (t, 2H, J=7.5 Hz); 2.05 to 1.85 (ms, 2H); 1.85 to 1.65 (ms, 2H); 1.65 to 1.45 (ms, 2H)

### Step b: Preparation of 1-isoindolin-2-yl-6-[4-(3-trifluoromethyl-phenyl)-piperazin-1-yl]hexan-1-one

A mixture of 1.2 g (4.05 mmol) of 2-(6-bromohexanoyl)isoindoline, 0.93 g (4.05 mmol) of 1-(3-trifluoromethylphenyl)piperazine, 15 mL of acetonitrile and 0.56 g (4.05 mmol) of potassium carbonate is brought to reflux for 18 hours.

After concentration of the acetonitrile, the residue is taken up with 20 mL of dichloromethane and washed twice with 15 mL of water. The organic phase is dried on magnesium sulphate, filtered and concentrated. The oily residue obtained is stirred with 15 mL of diisopropyl oxide for one hour. The precipitate is filtered and dried in a vacuum. In this way 1.5 g of 1-isoindolin-2-yl-6-[4-(3-trifluoromethylphenyl)piperazin-1-yl]hexan-1-one is obtained.

Melting point: 100-105° C.

NMR ¹H (CDCl₃): 7.4 to 7.2 (ms, 5H); 7.15 to 7.0 (ms, 3H); 4.8 (s, 4H); 3.3 to 3.15 (ms, 4H); 2.7 to 2.55 (ms, 4H); 2.5 to 2.3 (ms, 4H); 1.9 to 1.7 (ms, 2H); 1.7 to 1.5 (ms, 2H); 1.5 to 1.35 (ms, 2H)

### Step c: Preparation of hydrochloride of 1-isoindolin-2-yl-6-[4-(3-trifluoromethylphenyl)piperazin-1-yl]hexan-1-one

3 mL of a saturated solution of hydrogen chloride in diethyl oxide are added to a solution of 0.4 g (0.9 mmol) of 1-isoindolin-2-yl-6-[4-(3-trifluoromethylphenyl)piperazin-1-yl]hexan-1-one in 10 mL of acetone. After stirring for 15 minutes, the precipitate is filtered then dried in a vacuum on phosphorus pentoxide. In this way 0.37 g of 1-isoindolin-2-yl-6-[4-(3-trifluoromethylphenyl)piperazin-1-yl]-hexan-1-one hydrochloride is obtained.

Melting point: 165° C.

NMR ¹H (DMSO d₆): 11.1 (s wide, 1H); 7.3 (t, 1H, J=5 Hz); 7.4 to 7.2 (ms, 6H); 7.1 (d, 1H, J=5 Hz); 4.8 (s, 2H); 4.6 (s, 2H); 3.9 (d, 2H, J=8.5 Hz); 3.5 (d, 2H, J=8.5 Hz); 3.25 (t, 2H, J=7.5 Hz); 3.15 to 2.9 (ms, 4H); 2.35 (t, 2H, J=7.5 Hz); 1.9 to 1.7 (ms, 2H); 1.7 to 1.5 (ms, 2H); 1.45 to 1.25 (ms, 2H)

### Example 31

### Preparation of 1-isoindolin-2-yl-6-[4-(2-methoxyphenyl)piperazin-1-yl]hexan-1-one

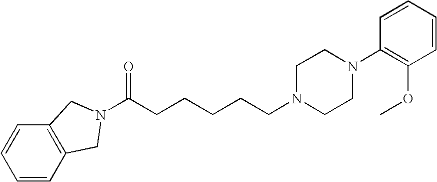

Operating as in Example 30, step b, but starting from 0.305 g of 2-(6-bromohexanoyl)isoindoline and 0.198 g of 1-(2-methoxyphenyl)piperazine, 0.205 g of 1-isoindolin-2-yl-6-[4-(2-methoxyphenyl)piperazin-1-yl]hexan-1-one is obtained in the form of a beige solid.

Melting point: 100.5° C.

NMR ¹H (CDCl₃): 7.4 to 7.2 (ms, 4H); 7.1 to 6.8 (ms, 4H); 4.8 (s, 4H); 3.8 (s, 3H); 3.25 to 3.0 (ms, 4H); 2.8 to 2.55 (ms, 4H); 2.55 to 2.3 (ms, 4H); 1.9 to 1.5 (ms, 4H); 1.5 to 1.3 (ms, 2H)

### Example 32

### Preparation of 1-isoindolin-2-yl-6-[4-(6,7,8,9-tetrahydro-5H-benzocylohepten-1-yl)piperazin-1-yl]hexan-1-one

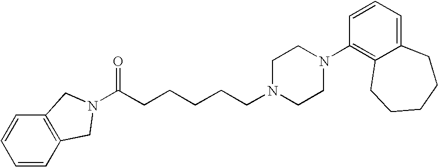

### Step a: Preparation of 6,7,8,9-tetrahydro-5H-benzocyclohepten-1-ylamine

Operating as in Example 5, step a, but starting from 0.5 g of 1-nitro-6,7,8,9-tetrahydro-5H-benzocycloheptene, 0.43 g of 6,7,8,9-tetrahydro-5H-benzocyclohepten-1-ylamine is obtained which are used as such in the subsequent steps.

The 1-nitro-6,7,8,9-tetrahydro-5H-benzocycloheptene can be obtained according to the process described in J. Am. Chem. Soc. (1969), 91, 3558-3566.

### Step b: Preparation of 1-(6,7,8,9-tetrahydro-5H-benzocyclohepten-2-yl)piperazine

Operating as in Example 5, step b, but starting from 0.43 g of 1-(6,7,8,9-tetrahydro-5H-benzocyclohepten-2-yl)amine and 0.48 g of bis(2-chloroethyl)amine, 0.54 g of 1-(6,7,8,9-tetrahydro-5H-benzocyclohepten-2-yl)piperazine is obtained.

Rf: 0.43 dichloromethane/methanol/ammonia 90/10/0.1

7.05 (d, 1H, J=7.5 Hz); 6.95 (s, 1H); 6.9 (t, 1H, J=7.5 Hz); 3.15 to 2.9 (ms, 8H); 2.9 to 2.7 (ms, 4H); 1.95 to 1.75 (ms, 2H); 1.75 to 1.5 (ms, 4H)

### Step c: Preparation of 1-isoindolin-2-yl-6-[4-(6,7,8,9-tetrahydro-5H-benzo-cylohepten-1-yl)piperazin-1-yl]hexan-1-one

Operating as in Example 30, step b, but starting from 191.5 mg of 1-(6,7,8,9-tetrahydro-5H-benzocyclohepten-2-yl)piperazine and 246.7 mg of 2-(6-bromohexanoyl)isoindoline, 106 mg of 1-isoindolin-2-yl-6-[4-(6,7,8,9-tetrahydro-5H-benzocylohepten-1-yl)piperazin-1-yl]hexan-1-one is obtained in the form of a cream solid.

Melting point: 131° C.

NMR ¹H (CDCl₃): 7.35 to 7.2 (ms, 4H); 7.15 to 6.8 (ms, 3H); 4.8 (s, 4H); 3.0 to 2.85 (ms, 4H); 3.85 to 2.5 (ms, 6H); 2.5 to 2.2 (ms, 6H); 1.95 to 1.7 (ms, 4H); 1.7 to 1.5 (ms, 6H); 1.5 to 1.3 (ms, 2H)

### Example 33

### Preparation of 1-isoindolin-2-yl-6-[4-(2,3-dimethylphenyl)-piperazin-1-yl]hexan-1-one

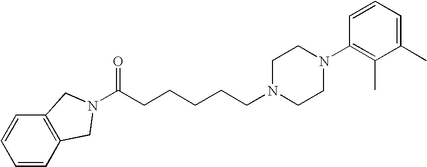

Operating as in Example 30, step b, but starting from 0.20 g of 1-(2,3-dimethylphenyl)piperazine and 0.30 g of 2-(6-bromohexanoyl)isoindoline, 0.28 g of 1-isoindolin-2-yl-6-[4-(2,3-dimethylphenyl)piperazin-1-yl]hexan-1-one is obtained in the form of a beige solid.

Melting point: 114° C.

NMR ¹H (CDCl₃): 7.4 to 7.2 (ms, 4H); 7.2 to 7.0 (ms, 1H); 7.0 to 6.8 (ms, 2H); 4.8 (s, 4H); 3.05 to 2.8 (ms, 4H); 2.8 to 2.5 (ms, 4H); 2.5 to 2.35 (ms, 4H); 2.3 (s, 3H); 2.1 (s, 3H); 1.9 to 1.7 (ms, 2H); 1.7 to 1.55 (ms, 2H); 1.55 to 1.3 (ms, 2H)

### Example 34

### Preparation of 1-isoindolin-2-yl-6-[4-benzofuran-7-ylpiperazin-1-yl]hexan-1-one

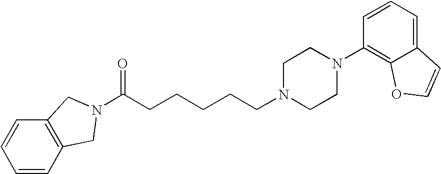

Operating as in Example 30, step b, but starting from 0.102 g of 1-benzofuran-7-ylpiperazine and 0.150 g of 2-(6-bromohexanoyl)isoindoline, 41 mg of 1-isoindolin-2-yl-6-[4-benzofuran-7-ylpiperazin-1-yl]hexan-1-one is obtained in the form of a whitish solid.

Melting point: 90° C.

NMR ¹H (CDCl₃): 7.6 (d, 1H, J=2.5 Hz); 7.4 to 7.1 (ms, 6H); 6.8 to 6.7 (ms, 2H); 4.8 (s, 4H); 3.5 to 3.25 (ms, 4H); 2.85 to 2.6 (ms, 4H); 2.6 to 2.35 (ms, 4H); 1.9 to 1.55 (ms, 4H); 1.55 to 1.35 (ms, 2H)

The 1-benzofuran-7-ylpiperazine can be prepared according to the process described in J. Med. Chem. (1988), 31, 1934-1940.

### Example 35

### Preparation of 1-isoindolin-2-yl-6-[4-(2-cyano-3-trifluoromethylphenyl)piperazin-1-yl]hexan-1-one

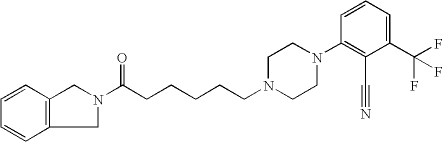

### Step a: Preparation of 1-(2-cyano-3-trifluoromethylphenyl)piperazine

A mixture of 3 g (15.8 mmol) of 2-fluoro-6-trifluoromethylbenzonitrile, 7.5 g (87 mmol) of piperazine and 24 mL of dioxan is heated to reflux for 5 hours.

The reaction medium is concentrated in a vacuum and the residue is taken up with ethyl acetate. After washing with water, the organic phase is dried on magnesium sulphate, filtered and concentrated. The product crystallises at ambient temperature. After drying in a vacuum, 3.6 g of 1-(2-cyano-3-trifluoromethylphenyl)piperazine is obtained which is used as such in the subsequent syntheses.

Rf: 0.65 dichloromethane/methanol/ammonia 90/10/1

### Step b: Preparation of 1-isoindolin-2-yl-6-[4-(2-cyano-3-trifluoromethylphenyl)piperazin-1-yl]hexan-1-one

Operating as in Example 30, step b, but starting from 0.14 g of 1-(2-cyano-3-trifluoromethylphenyl)piperazine and 0.15 g of 2-(6-bromobutanoyl)-isoindoline, 0.12 g of 1-isoindolin-2-yl-6-[4-(2-cyano-3-trifluoromethyl-phenyl)piperazin-1-yl]hexan-1-one is obtained in the form of a solid.

Melting point: 160° C.

NMR ¹H (CDCl₃): 7.5 (t, 1H, J=8.5 Hz); 7.4 to 7.15 (ms, 6H); 4.8 (s, 4H); 3.4 to 3.2 (ms, 4H); 2.8 to 2.6 (ms, 4H); 2.55 to 2.3 (ms 4H); 1.9 to 1.7 (ms, 2H); 1.7 to 1.5 (ms, 2H); 1.5 to 1.35 (ms, 2H)

### Example 36

### Preparation of 1-isoindolin-2-yl-6-(4-indan-5ylpiperazin-1-yl)hexan-1-one

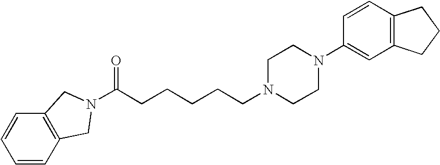

Operating as in Example 30, step b, but starting from 0.20 g of 1-(indan-5-yl)piperazine and 0.30 g of 2-(6-bromobutanoyl)isoindoline, 0.13 g of 1-isoindolin-2-yl-6-(4-indan-5ylpiperazin-1-yl)hexan-1-one is obtained in the form of a white solid.

Melting point: 131° C.

NMR ¹H (CDCl₃): 7.4 to 7.2 (ms, 4H); 7.1 (m, 1H); 6.85 (s, 1H); 6.75 (m, 1H); 4.8 (s, 4H); 3.2 (m, 4H); 2.9 (m, 4H); 2.6 (m, 4H); 2.5 to 2.35 (ms, 4H); 2.1 (m, 2H); 1.8 (m, 2H); 1.6 (m, 2H); 1.5 (m, 2H)

### Example 37

### Preparation of 1-isoindolin-2-yl-6-(4-indan-4ylpiperazin-1-yl)hexan-1-one

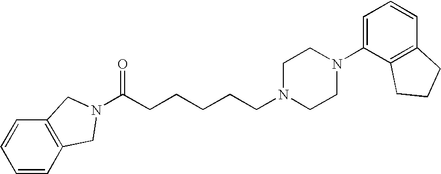

Operating as in Example 30, step b, but starting from 0.21 g of 1-(indan-4-yl)piperazine and 0.30 g of 2-(6-bromobutanoyl)isoindoline, 0.27 g of 1-isoindolin-2-yl-6-(4-indan-4ylpiperazin-1-yl)hexan-1-one is obtained in the form of a beige solid.

Melting point: 112° C.

NMR ¹H (CDCl₃): 7.4 to 7.2 (ms, 4H); 7.1 (m, 1H); 6.9 (m, 1H); 6.7 (m, 1H); 4.8 (s, 4H); 3.0 (m, 4H); 2.85 (m, 4H); 2.6 (m, 4H); 2.5 to 2.3 (ms, 4H); 2.1 (m, 2H); 1.8 (m, 2H); 1.7 to 1.55 (m, 2H); 1.45 (m, 2H)

### Example 38

### Preparation of 1-isoindolin-2-yl-6-[4-(5,6,7,8-tetrahydro-naphthalen-1-yl)piperazin-1-yl]hexan-1-one

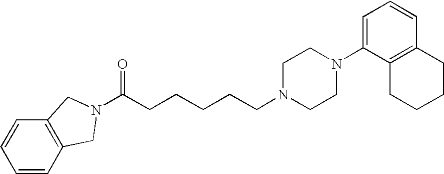

Operating as in Example 30, step c, but starting from 0.16 g of 1-(5,6,7,8-tetrahydronaphtalen-1-yl)piperazine and 0.22 g of 2-(6-bromobutanoyl)-isoindoline, 0.17 g of 1-isoindolin-2-yl-6-[4-(5,6,7,8-tetrahydro-naphtalen-1-yl)piperazin-1-yl]hexan-1-one is obtained in the form of a beige solid.

Melting point: 115° C.

NMR ¹H (CDCl₃): 7.4 to 7.2 (ms, 4H); 7.1 (m, 1H); 6.9 (s, 1H); 6.85 (m, 1H); 4.8 (s, 4H); 2.95 (m, 4H); 2.85 (m, 2H); 2.75 (m, 2H); 2.6 (m, 4H); 2.5 to 2.3 (ms, 4H); 1.9 to 1.55 (ms, 8H); 1.45 (m, 2H)

### Example 39

### Preparation of 1-isoindolin-2-yl-6-[4-(3-methanesulphonyloxyphenyl)piperazin-1-yl]hexan-1-one

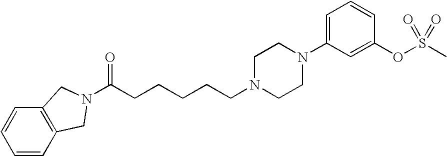

### Step a: Preparation of 1-isoindolin-2-yl-6-[4-(3-hydroxyphenyl)piperazin-1-yl]hexan-1-one

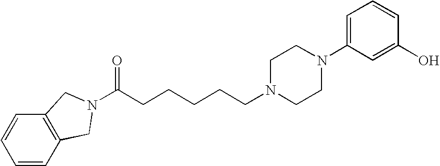

Operating as in Example 30, step b, but starting from 0.36 g of 1-(3-hydroxyphenyl)piperazine and 0.60 g of 2-(6-bromobutanoyl)isoindoline, 0.21 g of 1-isoindolin-2-yl-6-[4-(3-hydroxyphenyl)piperazin-1-yl]hexan-1-one is obtained in the form of a solid.

NMR ¹H (DMSO D₆): 9.0 (s, 1H); 7.4 to 7.2 (ms, 4H); 6.95 (m, 1H); 6.3 (m, 1H); 6.2 (s, 1H); 6.15 (m, 1H); 4.8 (s, 2H); 4.6 (s, 2H); 3.0 (m, 4H); 2.45 (m, 4H); 2.4 to 2.2 (ms, 4H); 1.6 (m, 2H); 1.45 (m, 2H); 1.3 (m, 2H)

### Step b: Preparation of 1-isoindolin-2-yl-6-[4-(3-methanesulphonyl-oxyphenyl)piperazin-1-yl]hexan-1-one

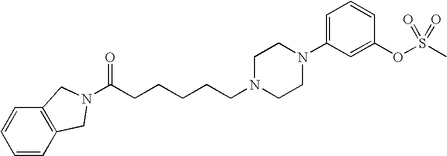

A solution of 0.2 g (0.51 mmol) of 1-isoindolin-2-yl-6-[4-(3-hydroxy-phenyl)piperazin-1-yl]hexan-1-one and 56 mg (0.56 mmol) of triethylamine in 5 mL of dichloromethane is cooled to 0° C. 64 mg (0.56 mmol) of methane sulphonyl chloride are then added.

The mixture is stirred during one night at ambient temperature, diluted with dichloromethane and washed three times with water. The organic phase is dried on magnesium sulphate, filtered and concentrated.

The oily residue is chromatographed on silica gel (dichloromethane/methanol ratio 98/2 to 95/5). In this way 75 mg (31%) of 1-isoindolin-2-yl-6-[4-(3-methanesulphonyloxyphenyl)piperazin-1-yl]hexan-1-one is obtained in the form of a beige solid.

Melting point: 141° C.

NMR ¹H (CDCl₃): 7.4 to 7.2 (ms, 5H); 6.85 (m, 1H); 6.8 (s, 1H); 6.7 (m, 1H); 4.8 (s, 4H); 3.25 (m, 4H); 3.15 (s, 3H); 2.6 (m, 4H); 2.5 to 2.3 (ms, 4H); 1.8 (m, 2H); 1.6 (m, 2H); 1.45 (m, 2H)

### Example 40

### Preparation of 1-isoindolin-2-yl-6-[4-(3,4-dihydro-2H-benzo[b][1,4]dioxepin-6-yl)piperazin-1-yl]hexan-1-one

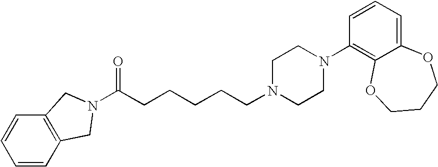

Operating as in Example 30, step b, but starting from 0.22 g of 1-(3,4-dihydro-2H-benzo[b][1,4]dioxepin-6-yl)piperazine and 0.17 g of 2-(6-bromo-butanoyl)isoindoline, 60 mg of 1-isoindolin-2-yl-6-[4-(3,4-dihydro-2H-benzo[b][1,4]dioxepin-6-yl)piperazin-1-yl]hexan-1-one is obtained in the form of a beige solid.

Melting point: 131° C.

NMR ¹H (CDCl₃): 7.4 to 7.2 (ms, 4H); 6.85 (m, 1H); 6.7 to 6.55 (ms, 2H); 4.8 (s, 4H); 4.25 (m, 4H); 3.1 (m, 4H); 2.6 (m, 4H); 2.5 to 2.35 (ms, 4H); 2.2 (m, 2H); 1.8 (m, 2H); 1.6 (m, 2H); 1.45 (m, 2H)

### Example 41

### Preparation of 1-isoindolin-2-yl-6-[4-(3-trifluoromethyl-5-cyanophenyl)piperazin-1-yl]hexan-1-one

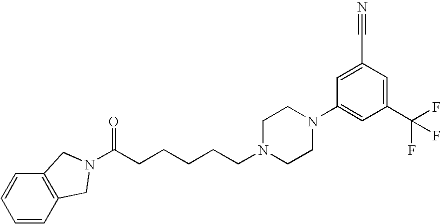

Operating as in Example 30, step b, but starting from 0.25 g of 1-(3-trifluoromethyl-5-cyanophenyl)piperazine and 0.30 g of 2-(6-bromo-butanoyl)-isoindoline, 0.22 g of 1-isoindolin-2-yl-6-[4-(3-trifluoromethyl-5-cyanophenyl)piperazin-1-yl]hexan-1-one is obtained in the form of a solid.

Melting point: 128° C.

NMR ¹H (CDCl₃): 7.35 to 7.15 (ms, 7H); 4.8 (s, 4H); 3.3 (m, 4H); 2.6 (m, 4H); 2.5 to 2.3 (ms, 4H); 1.8 (m, 2H); 1.6 (m, 2H); 1.45 (m, 2H)

### Example 42

### Preparation of 1-isoindolin-2-yl-6-[4-(5,6,7,8-tetrahydro-naphthalen-2-yl)piperazin-1-yl]hexan-1-one

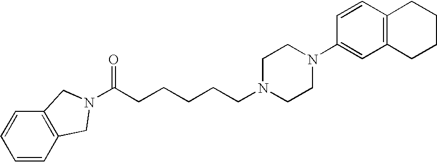

Operating as in Example 30, step b, but starting from 0.21 g of 1-(5,6,7,8-tetrahydronaphthalen-2-yl)piperazine and 0.29 g of 2-(6-bromobutanoyl)-isoindoline, 0.19 g of 1-isoindolin-2-yl-6-[4-(5,6,7,8-tetrahydro-naphthalen-2-yl)-piperazin-1-yl]hexan-1-one is obtained in the form of a beige solid.

Melting point: 123° C.

NMR ¹H (CDCl₃): 7.35 to 7.2 (ms, 3H); 7.0 (m, 1H); 6.75 (m, 2H); 6.65 (s, 1H); 4.8 (s, 4H); 3.15 (m, 4H); 2.75 (m, 4H); 2.6 (m, 4H); 2.5 to 2.35 (ms, 4H); 1.85 to 1.7 (ms, 6H); 1.6 (m, 2H); 1.45 (m, 2H)

### Example 43

### Preparation of 1-isoindolin-2-yl-6-[4-(3-isopropoxyphenyl)-piperazin-1-yl]hexan-1-one

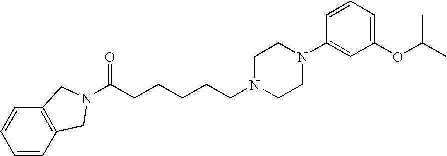

Operating as in Example 30, step b, but starting from 0.20 g of 1-(3-isopropoxyphenyl)piperazine and 0.30 g of 2-(6-bromobutanoyl)isoindoline, 0.29 g of 1-isoindolin-2-yl-6-[4-(3-isopropoxyphenyl)piperazin-1-yl]hexan-1-one is obtained in the form of a beige solid.

NMR ¹H (CDCl₃): 7.4 to 7.2 (ms, 4H); 7.15 (m, 1H); 6.6 to 6.45 (ms, 3H); 4.8 (s, 4H); 4.55 (m, 1H); 3.2 (m, 4H); 2.6 (m, 4H); 2.5 to 2.3 (ms, 4H); 1.8 (m, 2H); 1.6 (m, 2H); 1.45 (m, 2H); 1.3 (d, 6H, J=7 Hz)

### Example 44

### Preparation of 1-isoindolin-2-yl-6-[4-(2-cyano-3-methylphenyl)-piperazin-1-yl]hexan-1-one

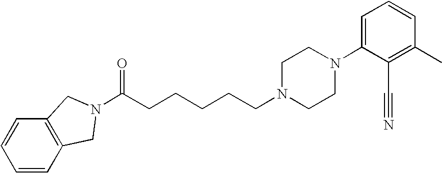

Operating as in Example 30, step b, but starting from 0.20 g of 1-(2-cyano-3-methylphenyl)piperazine and 0.30 g of 2-(6-bromobutanoyl)isoindoline, 0.21 g of 1-isoindolin-2-yl-6-[4-(2-cyano-3-methylphenyl)piperazin-1-yl]hexan-1-one is obtained in the form of a beige solid.

Melting point: 126° C.

NMR ¹H (CDCl₃): 7.4 to 7.2 (m, 5H); 6.95 to 6.75 (ms, 2H); 4.8 (s, 4H); 3.2 (m, 4H); 2.7 (m, 4H); 2.5 (s, 3H); 2.45 to 2.3 (ms, 4H); 1.8 (m, 2H); 1.6 (m, 2H); 1.45 (m, 2H)

### Example 45

### Preparation of 1-isoindolin-2-yl-6-[4-(2-cyano-3-isopropoxy)-piperazin-1-yl]hexan-1-one

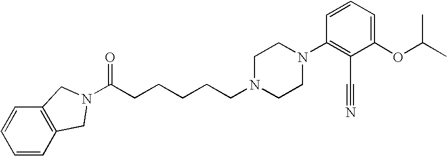

Operating as in Example 30, step b, but starting from 90 mg of 1-(2-cyano-3-isopropoxyphenyl)piperazine and 0.15 g of 2-(6-bromobutanoyl)-isoindoline, 66 mg of 1-isoindolin-2-yl-6-[4-(2-cyano-3-isopropoxy)-piperazin-1-yl]hexan-1-one is obtained in the form of a beige solid.

Melting point: 112° C.

NMR ¹H (CDCl₃): 7.4 to 7.2 (ms, 5H); 6.5 (m, 2H); 4.8 (s, 4H); 4.6 (m, 1H); 3.2 (m, 4H); 2.65 (m, 4H); 2.55 to 2.23 (ms, 4H); 1.8 (m, 2H); 1.6 (m, 2H); 1.45 (m, 2H); 1.4 (d, 6H, J=7 Hz)

### Examples 46 to 48

### Simultaneous Preparation of 1-(5,6-dimethoxyisoindolin-2-yl)-6-[4-(3-trifluoromethylphenyl)piperazin-1-yl]hexan-1-one, 1-isoindolin-2-yl-6-[4-(2-cyanophenyl)piperazin-1-yl]hexan-1-one and 1-isoindolin-2-yl-6-[4-(2-methylphenyl)piperazin-1-yl]hexan-1-one

### Step a: Preparation of 6-[4-(2-cyanophenyl)piperazin-1-yl]ethyl hexanoate

Operating as in Example 1, step a, but starting from 5 g of 1-(2-cyanophenyl)piperazine and 5.95 g of ethyl 6-bromohexanoate, 8.25 g of ethyl 6-[4-(2-cyanophenyl)piperazin-1-yl]hexanoate is obtained.

### Step b: Preparation of 6-[4-(2-cyanophenyl)piperazin-1-yl]hexanoic acid

Operating as in Example 1, step b, but starting from 8.25 g of 6-[4-(2-cyanophenyl)piperazin-1-yl]ethyl hexanoate, 6.83 g of 6-[4-(2-cyanophenyl)piperazin-1-yl]hexanoic acid is obtained in the form of a white solid.

Melting point: 120° C.

### Step c: Preparation of 6-[4-(2-methylphenyl)piperazin-1-yl]ethyl hexanoate

Operating as in Example 1, step a, but starting from 4.98 g of 1-(2-methylphenyl)piperazine dihydrochloride, 8.28 g of potassium carbonate and 4.46 g of 6-ethyl bromohexanoate, 6.3 g of 6-[4-(2-methylphenyl)piperazin-1-yl]ethyl hexanoate is obtained.

### Step d: Preparation of 6-[4-(2-methylphenyl)piperazin-1-yl]hexanoic acid

Operating as in Example 1, step b, but starting from 6.3 g of 6-[4-(2-methylphenyl)piperazin-1-yl]ethyl hexanoate, 3.35 g of 6-[4-(2-methylphenyl)piperazin-1-yl]hexanoic acid is obtained in two jets in the form of a white solid.

Melting point: 125° C.

### Step e: simultaneous preparation of 1-(5,6-dimethoxyisoindolin-2-yl)-6-[4-(3-trifluoromethyl-phenyl)piperazin-1-yl]hexan-1-one, 1-isoindolin-2-yl-6-[4-(2-cyanophenyl)piperazin-1-yl]hexan-1-one and 1-isoindolin-2-yl-6-[4-(2-methyl-phenyl)piperazin-1-yl]hexan-1-one

A mixture of 0.3 mmol of dicyclohexylcarbodiimide and 0.3 mmol of 1-hydroxybenzotriazole is added to a solution of acid (0.3 mmol) in 2 mL of anhydrous N,N-dimethylformamide. The mixture is left whilst stirring for four hours at 70° C. It is then cooled at ambient temperature, filtered in order to eliminate the dicyclohexylurea. Amine (0.2 mmol) is then added, then it is left for twenty hours at ambient temperature.

The N,N-dimethylformamide is evaporated out in a vacuum and the crude is taken up by 2 mL of dichloromethane. It is then washed twice with 1 mL of a normal aqueous solution of soda, then with 1 mL of water. The organic phase is dried then concentrated in a vacuum.

Starting from 6-[4-(3-trifluoromethyl-phenyl)piperazin-1-yl]hexanoic acid and 5,6-dimethoxyisoindoline, 1-(5,6-dimethoxyisoindolin-2-yl)-6-[4-(3-trifluoromethylphenyl)piperazin-1-yl]hexan-1-one is obtained in this way.

Rf: 0.76 dichloromethane/methanol 85/15

Starting from 6-[4-(2-cyanophenyl)piperazin-1-yl]hexanoic acid and isoindoline, 1-isoindolin-2-yl-6-[4-(2-cyanophenyl)piperazin-1-yl]hexan-1-one is obtained in this way.

Rf: 0.58 dichloromethane/methanol 90/10

Starting from 6-[4-(2-methyl-phenyl)piperazin-1-yl]hexanoic acid and isoindoline, 1-isoindolin-2-yl-6-[4-(2-methyl-phenyl)piperazin-1-yl]hexan-1-one is obtained in this way.

Rf: 0.58 dichloromethane/methanol 90/10

### Example 49

### Preparation of 1-isoindolin-2-yl-6-(4-phenyl-3,6-dihydro-2H-pyridin-1-yl)hexan-1-one

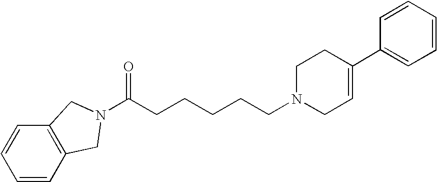

Operating as in Example 30, step b, but starting from 0.21 g of 4-phenyl-1,2,3,6-tetrahydropyridine and 0.30 g of 2-(6-bromobutanoyl)isoindoline, 0.17 g of 1-isoindolin-2-yl-6-[4-(phenyl-3,6-dihydro-2H-pyridin-1-yl]hexan-1-one is obtained in the form of a beige solid.

Melting point: 129° C.

NMR ¹H (CDCl₃): 7.45 to 7.15 (ms, 9H); 6.05 (m, 1H); 4.85 (s, 2H); 4.5 (s, 2H); 3.15 (m, 2H); 2.7 (m, 2H); 2.6 (m, 2H); 2.55 to 2.3 (4H); 1.8 (m, 2H); 1.6 (m, 2H); 1.45 (m, 2H)

### Example 50

### Preparation of 1-isoindolin-2-yl-6-[4-(3-trifluoromethylphenyl)-3,6-dihydro-2H-pyridin-1-yl]hexan-1-one

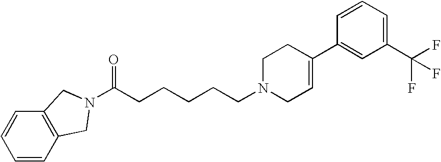

Operating as in Example 30, step b, but starting from 0.13 g of 4-(3-trifluoromethylphenyl)-1,2,3,6-tetrahydropyridine and 0.15 g of 2-(6-bromobutanoyl)isoindoline, 0.10 g of 1-isoindolin-2-yl-6-[4-(3-trifluoro-methylphenyl-3,6-dihydro-2H-pyridin-1-yl]hexan-1-one is obtained in the form of a beige solid.

Melting point: 86° C.

NMR ¹H (CDCl₃): 7.65 (s, 1H); 7.6 to 7.35 (ms, 3H); 7.35 to 7.2 (ms, 4H); 6.15 (m, 1H); 4.8 (s, 4H); 3.2 (m, 2H); 2.7 (t, 2H, J=7 Hz); 2.6 (m, 2H); 2.55 to 2.35 (ms, 4H); 1.8 (m, 2H); 1.6 (m, 2H); 1.45 (m, 2H)

### Example 51

### Preparation of 1-isoindolin-2-yl-6-[4-(3-trifluoromethyl-phenyl)-piperidin-1-yl]hexan-1-one

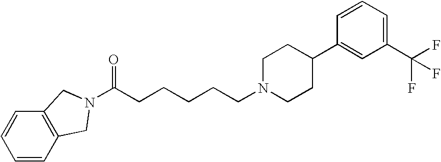

Operating as in Example 30, step b, but starting from 0.21 g of 4-(3-trifluoromethylphenyl)piperidine and 0.25 g of 2-(6-bromobutanoyl)isoindoline, 0.23 g of 1-isoindolin-2-yl-6-[4-(3-trifluoromethylphenyl)piperidin-1-yl]hexan-1-one is obtained in the form of a light grey solid.

Melting point: 71° C.

NMR ¹H (CDCl₃): 7.55 to 7.35 (ms, 4H); 7.35 to 7.2 (ms, 4H); 4.8 (s, 4H); 3.1 (m, 2H); 2.6 (m, 1H); 2.4 (t, 2H, J=7 Hz); 2.1 (m, 2H); 1.95 to 1.7 (ms, 6H); 1.7 to 1.55 (ms, 4H); 1.45 (m, 2H)

### Example 52

### Preparation of 1-isoindolin-2-yl-6-[4-(2-cyano-3-methylphenyl)-3,6-dihydro-2H-pyridin-1-yl]hexan-1-one

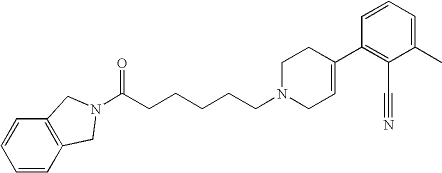

### Step a: Preparation of tert-butyl 4-(2-cyano-3-methylphenyl)-3,6-dihydro-2H-pyridin-1-carboxylate

A solution of 1.5 g (7.6 mmol) of 2-bromo-6-methylbenzonitrile in 10 mL of tetrahydrofuran is added at −65° C. to a solution of 4 mL of butyllithium 2.5M in hexane. After stirring for thirty minutes at −78° C., a solution of 16 mL (8 mmol) of 0.5 M zinc chloride in tetrahydrofuran is added. The stirring is continued for 90 minutes whilst the temperature is allowed to rise. The reaction medium is cooled to −10° C. 0.24 g (0.31 mmol) of palladium tetrakistriphenylphosphine is added followed by an addition of 1.7 g (5 mmol) of tert-butyl 4-trifluoromethanesulphonyloxy-3,6-dihydro-2H-pyridin-1-carboxylate (preparation described in J. Med. Chem., (2000), 43, 2703-2718) in 4 mL of tetrahydrofuran. The medium is heated to 40° C. for one hour.

After cooling at ambient temperature, the medium is poured onto 150 mL an aqueous sodium hydrogen carbonate solution. The product is extracted twice with ethyl acetate. The extraction phases are combined, dried on magnesium sulphate, filtered and concentrated. The product is purified by silica gel chromatography (eluent heptane/ethyl acetate 4/1). In this way 1.4 g (94%) of tert-butyl 4-(2-cyano-3-methylphenyl)-3,6-dihydro-2H-pyridin-1-carboxylate is obtained in the form of oil which crystallises at ambient temperature.

### Step b: Preparation of 4-(2-cyano-3-methylphenyl)-3,6-dihydro-2H-pyridine hydrochloride

15 mL of a saturated solution of hydrogen chloride in diethyl oxide are added to a solution of 1.4 g (4.7 mmol) of tert-butyl 4-(2-cyano-3-methylphenyl)-3,6-dihydro-2H-pyridin-1-carboxylate in 15 mL of ethyl acetate cooled to 0° C. The stirring is continued during one night at ambient temperature.

The precipitate is filtered, washed with diethyl oxide and dried in a vacuum. In this way 0.88 g (81%) of 4-(2-cyano-3-methylphenyl)-3,6-dihydro-2H-pyridine hydrochloride is obtained.

### Step c: Preparation of 1-isoindolin-2-yl-6-[4-(2-cyano-3-methylphenyl-3,6-dihydro-2H-pyridin-1-yl]hexan-1-one

Operating as in Example 30, step b, but starting from 0.40 g of 4-(2-cyano-3-methylphenyl)-3,6-dihydro-2H-pyridine hydrochloride and 0.50 g of 2-(6-bromobutanoyl)isoindoline, 0.57 g of 1-isoindolin-2-yl-6-[4-(2-cyano-3-methylphenyl)-3,6-dihydro-2H-pyridin-1-yl]hexan-1-one is obtained in the form of a beige solid.

Melting point: 71° C.

NMR ¹H (CDCl₃): 7.4 (m, 1H); 7.35 to 7.2 (ms, 4H); 7.15 (m, 2H); 6.0 (m, 1H); 4.8 (s, 4H); 3.2 (m, 2H); 2.75 (t, 2H, J=5 Hz); 2.65 to 2.5 (ms, 7H); 2.4 (t, 2H, J=7 Hz); 1.8 (m, 2H); 1.6 (m, 2H); 1.45 (m, 2H)

### Example 53

### Preparation of 1-isoindolin-2-yl-6-[4-(2-cyano-3-methylphenyl)-piperidin-1-yl]hexan-1-one

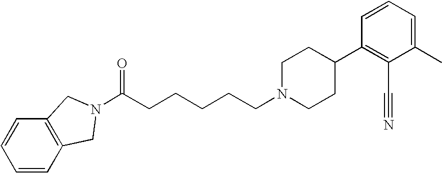

A mixture of 0.27 g (0.65 mmol) of 1-isoindolin-2-yl-6-[4-(2-cyano-3-methylphenyl)-3,6-dihydro-2H-pyridin-1-yl]hexan-1-one, 10 mL of methanol, 25 μL of acetic acid and 0.14 g of palladium on 10 carbon is hydrogenated at atmospheric pressure and at ambient temperature during one night.

After filtration on celite and rinsing with dichloromethane, the filtrate is concentrated then triturated in petroleum ether. The solid is filtered and dissolved in water. The solution is basified with a normal aqueous solution of soda. The product is filtered, washed with water and dried in a vacuum. In this way 0.18 g of 1-isoindolin-2-yl-6-[4-(2-cyano-3-methylphenyl)piperidin-1-yl]hexan-1-one is obtained.

Melting point: 132° C.

NMR ¹H (CDCl₃): 7.4 (m, 1H); 7.35 to 7.1 (ms, 6H); 4.8 (s, 4H); 3.1 (m, 2H); 3.0 (m, 1H); 2.55 (s, 3H); 2.4 (m, 4H); 2.15 (m, 2H); 2.0 to 1.7 (ms, 6H); 1.6 (m, 2H); 1.45 (m, 2H)

### Biological Results: Determination of the Affinity of the Compounds for the Human Dopamine D3 Receptor

Binding of [³H]spiperone:

CHO cells were transfected by the gene coding for the human dopamine D3 receptor (hD3). The binding of [³H]spiperone (0.5 to 2 nM) takes place in the presence of 5 to 10 μg of membranous proteins in a medium containing 120 mM NaCl, 5 mM KCl, and 50 mM Tris HCL pH 7.4; an incubation of 60 minutes at ambient temperature is necessary. The non-specific binding is estimated in the presence of 5 μM of haloperidol. The non-transfected cells are devoid of any binding activity.

TABLE 1
Antagonistic effects (Ki, nM) on the hD3 receptors measured
by binding of [³H] spiperone

| | hD3 |
|---|---|
| Example No. | Ki (nM) |
| 1 | 0.9 |
| 3 | 2.6 |
| 7 | 1.05 |
| 10 | 1.17 |
| 12 | 0.85 |
| 24 | 1.21 |
| 38 | 0.84 |
| 50 | 0.93 |
| 51 | 19.65 |

### Comparative Example

WO 01/49769 described compounds exhibiting a phenyl group at the position 4 of piperazine, the said phenyl group being substituted by a halogen atom. The following result demonstrates that such a compound has a low affinity for the dopamine D3 receptor:

| Structure | Ki (nM) |
|---|---|
| 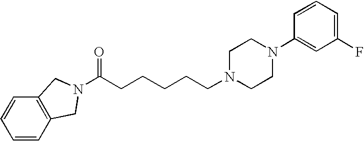 | 321 nM |

1. Compounds of formula (I) 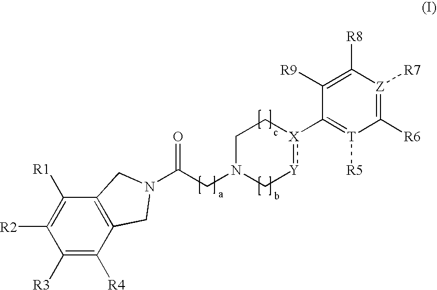
in which
a=5 or 6
b and c are identical or different and represent 1 or 2;
X [symbol] Y represents >N—CH₂—, >C═CH—, >CH—CH₂—;
Z and T are identical or different and independently represent a nitrogen or carbon atom; [symbol]
is absent or represents a simple bond, it being understood that when Z and/or T represents a nitrogen atom, [symbol] is absent;
R1, R2, R3 and R4 each independently represent a hydrogen atom or a halogen atom or a hydroxy, alkyl, monofluoroalkyl, polyfluoroalkyl, alkoxy, polyfluoroalkoxy, alkylsulphanyl, polyfluoroalkylsulphanyl, cyano, —OS(O)ₘ-alkyl, —NRR′, —COOR, —COR, —CONRR′, —C(OH)RR′, -alkylC(OH)RR′, -alkylCOOR, -alkylCOR, -alkylCONRR′, -alkylNRR′, -OalkylNRR′, -Oalkyl(NRR′)COOR group or saturated or unsaturated heterocycle or two of the adjacent R1, R2, R3 and R4 are joined to one another to form a hydrocarbon ring or a saturated or unsaturated heterocycle, fused to the phenyl core to which they are attached;
R5, R6, R7, R8 and R9 each independently represent a hydrogen atom or a hydroxy, alkyl, monofluoroalkyl, polyfluoroalkyl, alkoxy, polyfluoroalkoxy, alkylsulphanyl, polyfluoroalkylsulphanyl, cyano, —NRR′, —COOR, —COR, —CONRR′ group or two of the adjacent R5, R6, R7, R8 and R9 are linked to one another in order to form a hydrocarbon ring or a saturated or unsaturated heterocycle, fused to the phenyl core to which they are attached;
where R, R′, which are identical or different, independently represent a hydrogen atom, an alkyl group optionally substituted by one or several groups chosen from amongst OH, NRR′, OR, COOR or R and R′ are linked to one another in order to form a polymethylene or an oxapolymethylene;
m=0, 1 or 2;
it being understood that when T=Z=N, R5 and R7 are absent, R6 and R8 are chosen independently from amongst a hydrogen atom or an alkyl, monofluoroalkyl, polyfluoroalkyl, alkoxy, polyfluoroalkoxy, alkylsulphanyl, polyfluoroalkylsulphanyl, cyano, —COOR, —COR, —CONRR′ group and R9 is chosen from amongst a hydrogen atom or a hydroxy, alkyl, monofluoroalkyl, polyfluoroalkyl, alkoxy, polyfluoroalkoxy, alkylsulphanyl, polyfluoroalkylsulphanyl, —NRR′, —COOR, —COR, —CONRR′ group;
or R8 and R9 are linked to one another in order to form a hydrocarbon ring or a saturated or unsaturated heterocycle, fused to the phenyl core to which they are attached;
as well as their stereoisomers or mixtures, their tautomeric forms, their hydrates, solvates, their pharmaceutically acceptable salts and esters.

2. Compounds according to claim 1 in which a=5.

3. Compounds according to claim 1 in which b and c represent 1.

4. Compounds according to claim 1 in which [symbol] Y represents >N—CH₂—.

5. Compounds according to claim 1 in which Z=T=C or Z=T=N.

6. Compounds according to claim 1 in which R1, R2, R3, R4 independently represent a hydrogen or halogen atom or an alkoxy, —OS(O)ₘ-alkyl, cyano, —COOR, —COR, —CONRR′, —C(OH)RR′, -OalkylNRR′ group.

7. Compounds according to claim 1 in which when T=Z=C, R5, R6, R7, R8, R9 independently represent a hydrogen atom or an alkoxy, alkyl, cyano, polyfluoroalkoxy group, such as a perfluoroalkoxy group, such as trifluoromethoxy, or a polyfluoroalkyl group, such as a perfluoroalkyl group, such as trifluoromethyl, or two of the adjacent R5, R6, R7, R8 and R9 are linked to one another in order to form a hydrocarbon ring or a saturated or unsaturated heterocycle, fused to the phenyl core to which they are attached, such as a benzocycloheptene or benzofuran ring.

8. Compounds according to claim 1 in which when T=Z=N, R5 and R7 are absent, R6 and R8 are chosen independently from amongst a hydrogen atom or an alkyl, monofluoroalkyl, polyfluoroalkyl, alkoxy, polyfluoroalkoxy, alkylsulphanyl, polyfluoroalkylsulphanyl, cyano, —COOR, —COR, —CONRR′ group and R9 is chosen from amongst a hydrogen atom or a hydroxy, alkyl, monofluoroalkyl, polyfluoroalkyl, alkoxy, polyfluoroalkoxy, alkylsulphanyl, polyfluoroalkylsulphanyl, —NRR′, —COOR, —COR, —CONRR′ group or R8 and R9 are linked to one another in order to form a hydrocarbon ring or a saturated or unsaturated heterocycle, fused to the phenyl core to which they are attached.

9. Compounds according to claim 1 which are chosen from amongst the compounds of formula (I′): 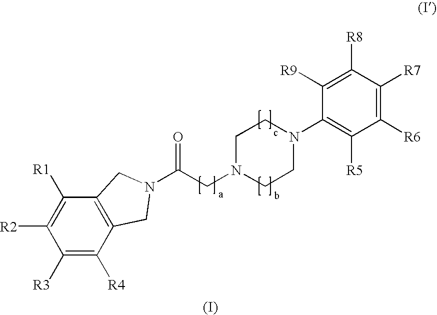
in which a, b and c, R1, R2, R3 and R4, R5, R6, R7, R8 and R9 are as defined in formula (I),
as well as their stereoisomers or mixtures, their tautomeric forms, their hydrates, solvates, their pharmaceutically acceptable salts and esters.

10. Compounds according to claim 1 which are chosen from amongst:
1-(5-methoxyisoindolin-2-yl)-6-[4-(3-trifluoromethylphenyl)piperazin-1-yl]hexan-1-one
1-(5-methanesulphonyloxyisoindolin-2-yl)-6-[4-(3-trifluoromethylphenyl)piperazin-1-yl]hexan-1-one
1-(5-cyanoisoindolin-2-yl)-6-[4-(3-trifluoromethylphenyl)-piperazin-1-yl]-hexan-1-one hydrochloride
1-isoindolin-2-yl-6-[4-(3-trifluoromethoxyphenyl)piperazin-1-yl]hexan-1-one
1-isoindolin-2-yl-6-[4-(6,7,8,9-tetrahydro-5H-benzocylohepten-2-yl)piperazin-1-yl]hexan-1-one
1-(4-fluoroisoindolin-2-yl)-6-[4-(3-trifluoromethylphenyl)piperazin-1-yl]hexan-1-one
1-(5-fluoroisoindolin-2-yl)-6-[4-(3-trifluoromethylphenyl)piperazin-1-yl]hexan-1-one
1-(5-isopropoxyisoindolin-2-yl)-6-[4-(3-trifluoromethylphenyl)piperazin-1-yl]hexan-1-one
1-(4-chloroisoindolin-2-yl)-6-[4-(3-trifluoromethylphenyl)piperazin-1-yl]hexan-1-one
1-(6,7-dihydro-5H-[1,3]dioxolo[4,5-f]isoindol-6-yl)-6-[4-(3-trifluoromethylphenyl)piperazin-1-yl]hexan-1-one
1-(5-hydroxymethylisoindolin-2-yl)-6-[4-(3-trifluoromethylphenyl)-piperazin-1-yl]hexan-1-one
1-(5-methoxycarbonylisoindolin-2-yl)-6-[4-(3-trifluoromethylphenyl)piperazin-1-yl]hexan-1-one hydrochloride
1-(5-carboxyisoindolin-2-yl)-6-[4-(3-trifluoromethylphenyl)piperazin-1-yl]hexan-1-one
1-[5-(2-hydroxyethylaminocarbonyl)isoindolin-2-yl]-6-[4-(3-trifluoromethylphenyl)piperazin-1-yl]hexan-1-one
1-(5-morpholinocarbonylisoindolin-2-yl)-6-[4-(3-trifluoromethylphenyl)-piperazin-1-yl]hexan-1-one
1-(5-(pyrrolidin-1-ylcarbonylisoindolin-2-yl)-6-[4-(3-trifluoromethylphenyl)piperazin-1-yl]hexan-1-one
1-(5-piperidin-1-ylcarbonylisoindolin-2-yl)-6-[4-(3-trifluoromethylphenyl)piperazin-1-yl]hexan-1-one
1-[5-(2-dimethylamino-ethylaminocarbonyl)isoindolin-2-yl]-6-[4-(3-trifluoromethylphenyl)piperazin-1-yl]hexan-1-one
1-[5-(4,5-dihydrooxazol-2yl)isoindolin-2-yl]-6-[4-(3-trifluoromethylphenyl)piperazin-1-yl]hexan-1-one
1-(5-morpholinoylmethylisoindolin-2-yl)-6-[4-(3-trifluoromethylphenyl)piperazin-1-yl]hexan-1-one hydrochloride
1-[5-(pyrrolidin-1-ylmethyl)isoindolin-2-yl]-6-[4-(3-trifluoromethylphenyl)piperazin-1-yl]hexan-1-one
1-[5-(dimethylaminomethyl)isoindolin-2-yl]-6-[4-(3-trifluoromethylphenyl)piperazin-1-yl]hexan-1-one
1-[5-(2-piperidinoethoxy)isoindolin-2-yl]-6-[4-(3-trifluoromethylphenyl)-piperazin-1-yl]hexan-1-one
1-[5-(3-piperidinopropoxy)isoindolin-2-yl]-6-[4-(3-trifluoromethylphenyl)piperazin-1-yl]hexan-1-one
1-[5-(3-pyrrolidinopropoxy)isoindolin-2-yl]-6-[4-(3-trifluoromethylphenyl)piperazin-1-yl]hexan-1-one
1-(5-acetyl isoindolin-2-yl)-6-[4-(3-trifluoro methylphenyl)piperazin-1-yl]hexan-1-one
1-[5-(1-hydroxy-1-methylethyl)isoindolin-2-yl]-6-[4-(3-trifluoromethylphenyl)piperazin-1-yl]hexan-1-one
1-[5-(1-hydroxyethyl)isoindolin-2-yl]-6-[4-(3-trifluoro-methylphenyl)piperazin-1-yl]hexan-1-one hydrochloride
1-(5-aminocarbonylisoindolin-2-yl)-6-[4-(3-trifluoromethylphenyl)-piperazin-1-yl]hexan-1-one
1-isoindolin-2-yl-6-[4-(3-trifluoromethylphenyl)-piperazin-1-yl]hexan-1-one hydrochloride
1-isoindolin-2-yl-6-[4-(2-methoxyphenyl)piperazin-1-yl]hexan-1-one
1-isoindolin-2-yl-6-[4-(6,7,8,9-tetrahydro-5H-benzocylohepten-1-yl)piperazin-1-yl]hexan-1-one
1-isoindolin-2-yl-6-[4-(2,3-dimethylphenyl)piperazin-1-yl]hexan-1-one
1-isoindolin-2-yl-6-[4-benzofuran-7-ylpiperazin-1-yl]hexan-1-one
1-isoindolin-2-yl-6-[4-(2-cyano-3-trifluoromethylphenyl)piperazin-1-yl]hexan-1-one
1-isoindolin-2-yl-6-(4-indan-5ylpiperazin-1-yl)hexan-1-one
1-isoindolin-2-yl-6-(4-indan-4ylpiperazin-1-yl)hexan-1-one
1-isoindolin-2-yl-6-[4-(5,6,7,8-tetrahydronaphthalen-1-yl)piperazin-1-yl]hexan-1-one
1-isoindolin-2-yl-6-[4-(3-methanesulphonyloxyphenyl)piperazin-1-yl]hexan-1-one
1-isoindolin-2-yl-6-[4-(3,4-dihydro-2H-benzo[b][1,4]dioxepin-6-yl)piperazin-1-yl]hexan-1-one
1-isoindolin-2-yl-6-[4-(3-trifluoromethyl-5-cyanophenyl)piperazin-1-yl]hexan-1-one
1-isoindolin-2-yl-6-[4-(5,6,7,8-tetrahydronaphthalen-2-yl)-piperazin-1-yl]hexan-1-one
1-isoindolin-2-yl-6-[4-(3-isopropoxyphenyl)piperazin-1-yl]hexan-1-one
1-isoindolin-2-yl-6-[4-(2-cyano-3-methylphenyl)piperazin-1-yl]hexan-1-one
1-isoindolin-2-yl-6-[4-(2-cyano-3-isopropoxy)piperazin-1-yl]hexan-1-one
1-(5,6-dimethoxyisoindolin-2-yl)-6-[4-(3-trifluoromethylphenyl)piperazin-1-yl]hexan-1-one
1-isoindolin-2-yl-6-[4-(2-cyanophenyl)piperazin-1-yl]hexan-1-one
1-isoindolin-2-yl-6-[4-(2-methyl-phenyl)piperazin-1-yl]hexan-1-one
1-isoindolin-2-yl-6-(4-phenyl-3,6-dihydro-2H-pyridin-1-yl)hexan-1-one
1-isoindolin-2-yl-6-[4-(3-trifluoromethylphenyl)-3,6-dihydro-2H-pyridin-1-yl]hexan-1-one
1-isoindolin-2-yl-6-[4-(3-trifluoromethylphenyl)piperidin-1-yl]hexan-1-one
1-isoindolin-2-yl-6-[4-(2-cyano-3-methylphenyl)-3,6-dihydro-2H-pyridin-1-yl]hexan-1-one
1-isoindolin-2-yl-6-[4-(2-cyano-3-methylphenyl)piperidin-1-yl]hexan-1-one,
as well as their stereoisomers or their mixtures, their tautomeric forms, their hydrates, solvates, their pharmaceutically acceptable salts, free forms and esters.

11. Compounds according to any claim 1 chosen from amongst:
1-(5-methoxyisoindolin-2-yl)-6-[4-(3-trifluoromethylphenyl)piperazin-1-yl]hexan-1-one
1-(5-cyanoisoindolin-2-yl)-6-[4-(3-trifluoromethylphenyl)-piperazin-1-yl]-hexan-1-one hydrochloride
1-(5-fluoroisoindolin-2-yl)-6-[4-(3-trifluoromethyl-phenyl)piperazin-1-yl]hexan-1-one
1-(6,7-dihydro-5H-[1,3]dioxolo[4,5-f]isoindol-6-yl)-6-[4-(3-trifluoromethylphenyl)piperazin-1-yl]hexan-1-one
1-(5-methoxycarbonylisoindolin-2-yl)-6-[4-(3-trifluoromethylphenyl)piperazin-1-yl]hexan-1-one hydrochloride
1-[5-(3-piperidinopropoxy)isoindolin-2-yl]-6-[4-(3-trifluoromethylphenyl)piperazin-1-yl]hexan-1-one
1-isoindolin-2-yl-6-[4-(5,6,7,8-tetrahydro-naphthalen-1-yl)piperazin-1-yl]hexan-1-one
1-isoindolin-2-yl-6-[4-(3-trifluoromethylphenyl)-3,6-dihydro-2H-pyridin-1-yl]hexan-1-one
1-isoindolin-2-yl-6-[4-(3-trifluoromethyl-phenyl)piperidin-1-yl]hexan-1-one
as well as their stereoisomers or their mixtures, their tautomeric forms, their hydrates, solvates, their pharmaceutically acceptable salts, free forms and esters.

12. Process of preparation of a compound of formula (I) according to claim 1 such that the process comprises the step of coupling of the compounds of formula (II) and (III): 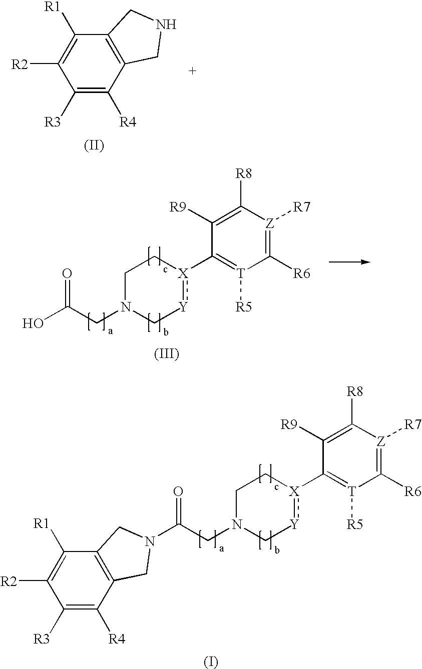
where, in the formula (II) and (III), R1, R2, R3, R4, R5, R6, R7, R8, R9, X, Y, Z, T, . . . , [symbol] , a, b and c have the same meaning as in formula (I).

13. Process of preparation of a compound of formula (I) according to claim 1 such that the process comprises the step of coupling compounds having the formula (VI) in which R5, R6, R7, R8, R9, X, Y, Z, T, . . . , [symbol] , b and c have the same meaning as in formula (I) and halogenated derivatives of formula (X) in which R1, R2, R3, R4 and a have the same meaning as in formula (I) and Hal represents a halogen:

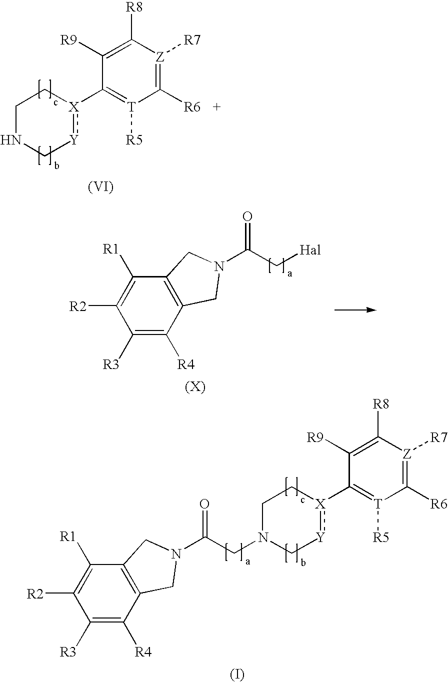

14. Process according to claim 12 in which the said process further comprises the subsequent step of isolation of the products of formula (I) which are obtained.

15. Pharmaceutical composition, characterised in that it comprises a therapeutically effective quantity of at least one compound according to claim 1 with a pharmaceutically acceptable vehicle or excipient.

16. Use of a compound of general formula (I) according to claim 1 for the preparation of pharmaceutical compositions intended to act as dopamine D3 receptor ligands.

17. Use of a compound of general formula (I) according to claim 1 for the preparation of pharmaceutical compositions intended to prevent and/or treat a neuropsychiatric disease.

18. Use of a compound of general formula (I) according to claim 1 for the preparation of pharmaceutical compositions intended to prevent and/or treat diseases causing the intervention of the dopamine D3 receptor.

19. Use according to claim 18 in which the said disease is chosen from amongst drug dependency, sexual problems, motor problems, Parkinson's disease, psychosis or a psychotic state, depression or drug dependency.

20. Use according to claim 19 in which the said drug dependency comprises any state linked to the breaking of a habit, abstinence and/or to the detoxification of a subject dependent upon any agent, in particular therapeutically active agents, such as morphine derivatives, and/or drugs such as cocaine, heroin, or even alcohol and/or nicotine.

21. Use according to claim 19 in which the said sexual problems comprise impotence, in particular male impotence.

22. Use according to claim 19 in which the said prevention and/or treatment of Parkinson's disease is a complementary treatment of Parkinson's disease.

23. Use according to claim 19, in which the said motor problems comprise essential or iatrogenic dyskinesias, and/or essential or iatrogenic tremors.
